SEQUENCE LISTING (22 sequences)

SEQ ID NO: 1 - DNA, 3915 bp, Clostridium botulinum
atggcatttgttaataaacaatttaattataaagatcctgtaaatggtgttgatattgcttatataaaaattccaaatgcaggacaaatgcaaccagtaaaagcttttaaaattcataataaaatatgggttattccagaaagagatacatttacaaatcctgaagaaggagatttaaatccaccaccagaagcaaaacaagttccagtttcatattatgattcaacatatttaagtacagataatgaaaaagataattatttaaagggagttacaaaattatttgagagaatttattcaactgatcttggaagaatgttgttaacatcaatagtaaggggaataccattttggggtggaagtacaatagatacagaattaaaagttattgatactaattgtattaatgtgatacaaccagatggtagttatagatcagaagaacttaatctagtaataataggaccctcagctgatattatacagtttgaatgtaaaagctttggacatgaagttttgaatcttacgcgaaatggttatggctctactcaatacattagatttagcccagattttacatttggttttgaggagtcacttgaagttgatacaaatcctcttttaggtgcaggcaaatttgctacagatccagcagtaacattagcacatgaacttatacatgctggacatagattatatggaatagcaattaatccaaatagggtttttaaagtaaatactaatgcctattatgaaatgagtgggttagaagtaagctttgaggaacttagaacatttgggggacatgatgcaaagtttatagatagtttacaggaaaacgaatttcgtctatattattataataagtttaaagatatagcaagtacacttaataaagctaaatcaatagtaggtactactgcttcattacagtatatgaaaaatgtttttaaagagaaatatctcctatctgaagatacatctggaaaattttcggtagataaattaaaatttgataagttatacaaaatgttaacagagatttacacagaggataattttgttaagttttttaaagtacttaacagaaaaacatatttgaattttgataaagccgtatttaagataaatatagtacctaaggtaaattacacaatatatgatggatttaatttaagaaatacaaatttagcagcaaactttaatggtcaaaatacagaaattaataatatgaattttactaaactaaaaaattttactggattgtttgaattttataagttgctatgtgtaagagggataataacttctaaaactaaatcattagttccgcgtggatccaaggccttaaatgatttatgtatcaaagttaataattgggacttgttttttagtccttcagaagataattttactaatgatctaaataaaggagaagaaattacatctgatactaatatagaagcagcagaagaaaatattagtttagatttaatacaacaatattatttaacctttaattttgataatgaacctgaaaatatttcaatagaaaatctttcaagtgacattataggccaattagaacttatgcctaatatagaaagatttcctaatggaaaaaagtatgagttagataaatatactatgttccattatcttcgtgctcaagaatttgaacatggtaaatctaggattgctttaacaaattctgttaacgaagcattattaaatcctagtcgtgtttatacatttttttcttcagactatgtaaagaaagttaataaagctacggaggcagctatgtttttaggctgggtagaacaattagtatatgattttaccgatgaaactagcgaagtaagtactacggataaaattgcggatataactataattattccatatataggacctgctttaaatataggtaatatgttatataaagatgattttgtaggtgctttaatattttcaggagctgttattctgttagaatttataccagagattgcaatacctgtattaggtacttttgcacttgtatcatatattgcgaataaggttctaaccgttcaaacaatagataatgctttaagtaaaagaaatgaaaaatgggatgaggtctataaatatatagtaacaaattggttagcaaaggttaatacacagattgatctaataagaaaaaaaatgaaagaagctttagaaaatcaagcagaagcaacaaaggctataataaactatcagtataatcaatatactgaggaagagaaaaataatattaattttaatattgatgatttaagttcgaaacttaatgagtctataaataaagctatgattaatataaataaatttttgaatcaatgctctgtttcatatttaatgaattctatgatcccttatggtgttaaacggttagaagattttgatgctagtcttaaagatgcattattaaagtatatatatgataatagaggaactttaattggtcaagtagatagattaaaagataaagttaataatacacttagtacagatataccttttcagctttccaaatacgtagataatcaaagattattatctacatttactgaatatattaagaatattattaatacttctatattgaatttaagatatgaaagtaatcatttaatagacttatctaggtatgcatcaaaaataaatattggtagtaaagtaaattttgatccaatagataaaaatcaaattcaattatttaatttagaaagtagtaaaattgaggtaattttaaaaaatgctattgtatataatagtatgtatgaaaattttagtactagcttttggataagaattcctaagtattttaacagtataagtctaaataatgaatatacaataataaattgtatggaaaataattcaggatggaaagtatcacttaattatggtgaaataatctggactttacaggatactcaggaaataaaacaaagagtagtttttaaatacagtcaaatgattaatatatcagattatataaacagatggatttttgtaactatcactaataatagattaaataactctaaaatttatataaatggaagattaatagatcaaaaaccaatttcaaatttaggtaatattcatgctagtaataatataatgtttaaattagatggttgtagagatacacatagatatatttggataaaatattttaatctttttgataaggaattaaatgaaaaagaaatcaaagatttatatgataatcaatcaaattcaggtattttaaaagacttttggggtgattatttacaatatgataaaccatactatatgttaaatttatatgatccaaataaatatgtcgatgtaaataatgtaggtattagaggttatatgtatcttaaagggcctagaggtagcgtaatgactacaaacatttatttaaattcaagtttgtatagggggacaaaatttattataaaaaaatatgcttctggaaataaagataatattgttagaaataatgatcgtgtatatattaatgtagtagttaaaaataaagaatataggttagctactaatgcatcacaggcaggcgtagaaaaaatactaagtgcattagaaatacctgatgtaggaaatctaagtcaagtagtagtaatgaagtcaaaaaatgatcaaggaataacaaataaatgcaaaatgaatttacaagataataatgggaatgatataggctttataggatttcatcagtttaataatatagctaaactagtagcaagtaattggtataatagacaaatagaaagatctagtaggactttgggttgctcatgggaatttattcctgtagatgatggatggggagaaaggccactgctcgagcaccaccaccaccaccactaa

SEQ ID NO: 2 - protein, 1304 aa, Clostridium botulinum
MAFVNKQFNYKDPVNGVDIAYIKIPNVGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLVPRGSKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPLLEHHHHHH

SEQ ID NO: 3 - DNA, 2646 bp, Clostridium botulinum
atggcatttgttaataaacaatttaattataaagatcctgtaaatggtgttgatattgcttatataaaaattccaaatgcaggacaaatgcaaccagtaaaagcttttaaaattcataataaaatatgggttattccagaaagagatacatttacaaatcctgaagaaggagatttaaatccaccaccagaagcaaaacaagttccagtttcatattatgattcaacatatttaagtacagataatgaaaaagataattatttaaagggagttacaaaattatttgagagaatttattcaactgatcttggaagaatgttgttaacatcaatagtaaggggaataccattttggggtggaagtacaatagatacagaattaaaagttattgatactaattgtattaatgtgatacaaccagatggtagttatagatcagaagaacttaatctagtaataataggaccctcagctgatattatacagtttgaatgtaaaagctttggacatgaagttttgaatcttacgcgaaatggttatggctctactcaatacattagatttagcccagattttacatttggttttgaggagtcacttgaagttgatacaaatcctcttttaggtgcaggcaaatttgctacagatccagcagtaacattagcacatgaacttatacatgctggacatagattatatggaatagcaattaatccaaatagggtttttaaagtaaatactaatgcctattatgaaatgagtgggttagaagtaagctttgaggaacttagaacatttgggggacatgatgcaaagtttatagatagtttacaggaaaacgaatttcgtctatattattataataagtttaaagatatagcaagtacacttaataaagctaaatcaatagtaggtactactgcttcattacagtatatgaaaaatgtttttaaagagaaatatctcctatctgaagatacatctggaaaattttcggtagataaattaaaatttgataagttatacaaaatgttaacagagatttacacagaggataattttgttaagttttttaaagtacttaacagaaaaacatatttgaattttgataaagccgtatttaagataaatatagtacctaaggtaaattacacaatatatgatggatttaatttaagaaatacaaatttagcagcaaactttaatggtcaaaatacagaaattaataatatgaattttactaaactaaaaaattttactggattgtttgaattttataagttgctatgtgtaagagggataataacttctaaaactaaatcattagttccgcgtggatccaaggccttaaatgatttatgtatcaaagttaataattgggacttgttttttagtccttcagaagataattttactaatgatctaaataaaggagaagaaattacatctgatactaatatagaagcagcagaagaaaatattagtttagatttaatacaacaatattatttaacctttaattttgataatgaacctgaaaatatttcaatagaaaatctttcaagtgacattataggccaattagaacttatgcctaatatagaaagatttcctaatggaaaaaagtatgagttagataaatatactatgttccattatcttcgtgctcaagaatttgaacatggtaaatctaggattgctttaacaaattctgttaacgaagcattattaaatcctagtcgtgtttatacatttttttcttcagactatgtaaagaaagttaataaagctacggaggcagctatgtttttaggctgggtagaacaattagtatatgattttaccgatgaaactagcgaagtaagtactacggataaaattgcggatataactataattattccatatataggacctgctttaaatataggtaatatgttatataaagatgattttgtaggtgctttaatattttcaggagctgttattctgttagaatttataccagagattgcaatacctgtattaggtacttttgcacttgtatcatatattgcgaataaggttctaaccgttcaaacaatagataatgctttaagtaaaagaaatgaaaaatgggatgaggtctataaatatatagtaacaaattggttagcaaaggttaatacacagattgatctaataagaaaaaaaatgaaagaagctttagaaaatcaagcagaagcaacaaaggctataataaactatcagtataatcaatatactgaggaagagaaaaataatattaattttaatattgatgatttaagttcgaaacttaatgagtctataaataaagctatgattaatataaataaatttttgaatcaatgctctgtttcatatttaatgaattctatgatcccttatggtgttaaacggttagaagattttgatgctagtcttaaagatgcattattaaagtatatatatgataatagaggaactttaattggtcaagtagatagattaaaagataaagttaataatacacttagtacagatataccttttcagctttccaaatacgtagataatcaaagattattatctacatttactgaatatattaagaatattctcgagcaccaccaccaccaccactaa

SEQ ID NO: 4 - protein, 881 aa, Clostridium botulinum
MAFVNKQFNYKDPVNGVDIAYIKIPNVGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLVPRGSKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNILEHHHHHH

SEQ ID NO: 5 - DNA, 3312 bp, Clostridium botulinum
atggcatttgttaataaacaatttaattataaagatcctgtaaatggtgttgatattgcttatataaaaattccaaatgcaggacaaatgcaaccagtaaaagcttttaaaattcataataaaatatgggttattccagaaagagatacatttacaaatcctgaagaaggagatttaaatccaccaccagaagcaaaacaagttccagtttcatattatgattcaacatatttaagtacagataatgaaaaagataattatttaaagggagttacaaaattatttgagagaatttattcaactgatcttggaagaatgttgttaacatcaatagtaaggggaataccattttggggtggaagtacaatagatacagaattaaaagttattgatactaattgtattaatgtgatacaaccagatggtagttatagatcagaagaacttaatctagtaataataggaccctcagctgatattatacagtttgaatgtaaaagctttggacatgaagttttgaatcttacgcgaaatggttatggctctactcaatacattagatttagcccagattttacatttggttttgaggagtcacttgaagttgatacaaatcctcttttaggtgcaggcaaatttgctacagatccagcagtaacattagcacatgaacttatacatgctggacatagattatatggaatagcaattaatccaaatagggtttttaaagtaaatactaatgcctattatgaaatgagtgggttagaagtaagctttgaggaacttagaacatttgggggacatgatgcaaagtttatagatagtttacaggaaaacgaatttcgtctatattattataataagtttaaagatatagcaagtacacttaataaagctaaatcaatagtaggtactactgcttcattacagtatatgaaaaatgtttttaaagagaaatatctcctatctgaagatacatctggaaaattttcggtagataaattaaaatttgataagttatacaaaatgttaacagagatttacacagaggataattttgttaagttttttaaagtacttaacagaaaaacatatttgaattttgataaagccgtatttaagataaatatagtacctaaggtaaattacacaatatatgatggatttaatttaagaaatacaaatttagcagcaaactttaatggtcaaaatacagaaattaataatatgaattttactaaactaaaaaattttactggattgtttgaattttataagttgctatgtgtaagagggataataacttctaaaactaaatcattagttccgcgtggatccaaggccttaaatgatttatgtatcaaagttaataattgggacttgttttttagtccttcagaagataattttactaatgatctaaataaaggagaagaaattacatctgatactaatatagaagcagcagaagaaaatattagtttagatttaatacaacaatattatttaacctttaattttgataatgaacctgaaaatatttcaatagaaaatctttcaagtgacattataggccaattagaacttatgcctaatatagaaagatttcctaatggaaaaaagtatgagttagataaatatactatgttccattatcttcgtgctcaagaatttgaacatggtaaatctaggattgctttaacaaattctgttaacgaagcattattaaatcctagtcgtgtttatacatttttttcttcagactatgtaaagaaagttaataaagctacggaggcagctatgtttttaggctgggtagaacaattagtatatgattttaccgatgaaactagcgaagtaagtactacggataaaattgcggatataactataattattccatatataggacctgctttaaatataggtaatatgttatataaagatgattttgtaggtgctttaatattttcaggagctgttattctgttagaatttataccagagattgcaatacctgtattaggtacttttgcacttgtatcatatattgcgaataaggttctaaccgttcaaacaatagataatgctttaagtaaaagaaatgaaaaatgggatgaggtctataaatatatagtaacaaattggttagcaaaggttaatacacagattgatctaataagaaaaaaaatgaaagaagctttagaaaatcaagcagaagcaacaaaggctataataaactatcagtataatcaatatactgaggaagagaaaaataatattaattttaatattgatgatttaagttcgaaacttaatgagtctataaataaagctatgattaatataaataaatttttgaatcaatgctctgtttcatatttaatgaattctatgatcccttatggtgttaaacggttagaagattttgatgctagtcttaaagatgcattattaaagtatatatatgataatagaggaactttaattggtcaagtagatagattaaaagataaagttaataatacacttagtacagatataccttttcagctttccaaatacgtagataatcaaagattattatctacatttactgaatatattaagaatattattaatacttctatattgaatttaagatatgaaagtaatcatttaatagacttatctaggtatgcatcaaaaataaatattggtagtaaagtaaattttgatccaatagataaaaatcaaattcaattatttaatttagaaagtagtaaaattgaggtaattttaaaaaatgctattgtatataatagtatgtatgaaaattttagtactagcttttggataagaattcctaagtattttaacagtataagtctaaataatgaatatacaataataaattgtatggaaaataattcaggatggaaagtatcacttaattatggtgaaataatctggactttacaggatactcaggaaataaaacaaagagtagtttttaaatacagtcaaatgattaatatatcagattatataaacagatggatttttgtaactatcactaataatagattaaataactctaaaatttatataaatggaagattaatagatcaaaaaccaatttcaaatttaggtaatattcatgctagtaataatataatgtttaaattagatggttgtagagatacacatagatatatttggataaaatattttaatctttttgataaggaattaaatgaaaaagaaatcaaagatttatatgataatcaatcaaattcaggtctcgagcaccaccaccaccaccactaa

SEQ ID NO: 6 - protein, 1103 aa, Clostridium botulinum
MAFVNKQFNYKDPVNGVDIAYIKIPNVGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLVPRGSKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGLEHHHHHH

SEQ ID NO: 7 - DNA, 3942 bp, Clostridium botulinum
atggcagttacaataaataattttaattataatgatcctattgataataataatattattatgatggagcctccatttgcgagaggtacggggagatattataaagcttttaaaatcacagatcgtatttggataataccggaaagatatacttttggatataaacctgaggattttaataaaagttccggtatttttaatagagatgtttgtgaatattatgatccagattacttaaatactaatgataaaaagaatatatttttacaaacaatgatcaagttatttaatagaatcaaatcaaaaccattgggtgaaaagttattagagatgattataaatggtataccttatcttggagatagacgtgttccactcgaagagtttaacacaaacattgctagtgtaactgttaataaattaatcagtaatccaggagaagtggagcgaaaaaaaggtattttcgcaaatttaataatatttggacctgggccagttttaaatgaaaatgagactatagatataggtatacaaaatcattttgcatcaagggaaggcttcgggggtataatgcaaatgaagttttgcccagaatatgtaagcgtatttaataatgttcaagaaaacaaaggcgcaagtatatttaatagacgtggatatttttcagatccagccttgatattaatgcatgaacttatacatgttttacatggattatatggcattaaagtagatgatttaccaattgtaccaaatgaaaaaaaattttttatgcaatctacagatgctatacaggcagaagaactatatacatttggaggacaagatcccagcatcataactccttctacggataaaagtatctatgataaagttttgcaaaattttagagggatagttgatagacttaacaaggttttagtttgcatatcagatcctaacattaatattaatatatataaaaataaatttaaagataaatataaattcgttgaagattctgagggaaaatatagtatagatgtagaaagttttgataaattatataaaagcttaatgtttggttttacagaaactaatatagcagaaaattataaaataaaaactagagcttcttattttagtgattccttaccaccagtaaaaataaaaaatttattagataatgaaatctatactatagaggaagggtttaatatatctgataaagatatggaaaaagaatatagaggtcagaataaagctataaataaacaagcttatgaagaaattagcaaggagcatttggctgtatataagatacaaatgtgtaaaagtgggataataacttctaaaactaaatcattggttccacgtggatccaaggcctcaggaatatgtattgatgttgataatgaagatttgttctttatagctgataaaaatagtttttcagatgatttatctaaaaacgaaagaatagaatataatacacagagtaattatatagaaaatgacttccctataaatgaattaattttagatactgatttaataagtaaaatagaattaccaagtgaaaatacagaatcacttactgattttaatgtagatgttccagtatatgaaaaacaacccgctataaaaaaaatttttacagatgaaaataccatctttcaatatttatactctcagacatttcctctagatataagagatataagtttaacatcttcatttgatgatgcattattattttctaacaaagtttattcatttttttctatggattatattaaaactgctaataaagtggtagaagcaggattatttgcaggttgggtgaaacagatagtaaatgattttgtaatcgaagctaataaaagcaatactatggataaaattgcagatatatctctaattgttccttatataggattagctttaaatgtaggaaatgaaacagctaaaggaaattttgaaaatgcttttgagattgcaggagccagtattctactagaatttataccagaacttttaatacctgtagttggagcctttttattagaatcatatattgacaataaaaataaaattattaaaacaatagataatgctttaactaaaagaaatgaaaaatggagtgatatgtacggattaatagtagcgcaatggctctcaacagttaatactcaattttatacaataaaagagggaatgtataaggctttaaattatcaagcacaagcattggaagaaataataaaatacagatataatatatattctgaaaaagaaaagtcaaatattaacatcgattttaatgatataaattctaaacttaatgagggtattaaccaagctatagataatataaataattttataaatggatgttctgtatcatatttaatgaaaaaaatgattccattagctgtagaaaaattactagactttgataatactctcaaaaaaaatttgttaaattatatagatgaaaataaattatatttgattggaagtgcagaatatgaaaaatcaaaagtaaataaatacttgaaaaccattatgccgtttgatctttcaatatataccaatgatacaatactaatagaaatgtttaataaatataatagcgaaattttaaataatattatcttaaatttaagatataaggataataatttaatagatttatcaggatatggggcaaaggtagaggtatatgatggagtcgagcttaatgataaaaatcaatttaaattaactagttcagcaaatagtaagattagagtgactcaaaatcagaatatcatatttaatagtgtgttccttgattttagcgttagcttttggataagaatacctaaatataagaatgatggtatacaaaattatattcataatgaatatacaataattaattgtatgaaaaataattcgggctggaaaatatctattaggggtaataggataatatggactttaattgatataaatggaaaaaccaaatcggtattttttgaatataacataagagaagatatatcagagtatataaatagatggttttttgtaactattactaataatttgaataacgctaaaatttatattaatggtaagctagaatcaaatacagatattaaagatataagagaagttattgctaatggtgaaataatatttaaattagatggtgatatagatagaacacaatttatttggatgaaatatttcagtatttttaatacggaattaagtcaatcaaatattgaagaaagatataaaattcaatcatatagcgaatatttaaaagatttttggggaaatcctttaatgtacaataaagaatattatatgtttaatgcggggaataaaaattcatatattaaactaaagaaagattcacctgtaggtgaaattttaacacgtagcaaatataatcaaaattctaaatatataaattatagagatttatatattggagaaaaatttattataagaagaaagtcaaattctcaatctataaatgatgatatagttagaaaagaagattatatatatctagatttttttaatttaaatcaagagtggagagtatatacctataaatattttaagaaagaggaagaaaaattgtttttagctcctataagtgattctgatgagttttacaatactatacaaataaaagaatatgatgaacagccaacatatagttgtcagttgctttttaaaaaagatgaagaaagtactgatgagataggattgattggtattcatcgtttctacgaatctggaattgtatttgaagagtataaagattatttttgtataagtaaatggtacttaaaagaggtaaaaaggaaaccatataatttaaaattgggatgtaattggcagtttattcctaaagatgaagggtggactgaactcgagcaccaccaccaccaccactaa

SEQ ID NO: 8 - protein, 1313 aa, Clostridium botulinum
MAVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSGIITSKTKSLVPRGSKASGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTELEHHHHHH

SEQ ID NO: 9 - DNA, 2649 bp, Clostridium botulinum
atggcagttacaataaataattttaattataatgatcctattgataataataatattattatgatggagcctccatttgcgagaggtacggggagatattataaagcttttaaaatcacagatcgtatttggataataccggaaagatatacttttggatataaacctgaggattttaataaaagttccggtatttttaatagagatgtttgtgaatattatgatccagattacttaaatactaatgataaaaagaatatatttttacaaacaatgatcaagttatttaatagaatcaaatcaaaaccattgggtgaaaagttattagagatgattataaatggtataccttatcttggagatagacgtgttccactcgaagagtttaacacaaacattgctagtgtaactgttaataaattaatcagtaatccaggagaagtggagcgaaaaaaaggtattttcgcaaatttaataatatttggacctgggccagttttaaatgaaaatgagactatagatataggtatacaaaatcattttgcatcaagggaaggcttcgggggtataatgcaaatgaagttttgcccagaatatgtaagcgtatttaataatgttcaagaaaacaaaggcgcaagtatatttaatagacgtggatatttttcagatccagccttgatattaatgcatgaacttatacatgttttacatggattatatggcattaaagtagatgatttaccaattgtaccaaatgaaaaaaaattttttatgcaatctacagatgctatacaggcagaagaactatatacatttggaggacaagatcccagcatcataactccttctacggataaaagtatctatgataaagttttgcaaaattttagagggatagttgatagacttaacaaggttttagtttgcatatcagatcctaacattaatattaatatatataaaaataaatttaaagataaatataaattcgttgaagattctgagggaaaatatagtatagatgtagaaagttttgataaattatataaaagcttaatgtttggttttacagaaactaatatagcagaaaattataaaataaaaactagagcttcttattttagtgattccttaccaccagtaaaaataaaaaatttattagataatgaaatctatactatagaggaagggtttaatatatctgataaagatatggaaaaagaatatagaggtcagaataaagctataaataaacaagcttatgaagaaattagcaaggagcatttggctgtatataagatacaaatgtgtaaaagtgggataataacttctaaaactaaatcattggttccacgtggatccaaggcctcaggaatatgtattgatgttgataatgaagatttgttctttatagctgataaaaatagtttttcagatgatttatctaaaaacgaaagaatagaatataatacacagagtaattatatagaaaatgacttccctataaatgaattaattttagatactgatttaataagtaaaatagaattaccaagtgaaaatacagaatcacttactgattttaatgtagatgttccagtatatgaaaaacaacccgctataaaaaaaatttttacagatgaaaataccatctttcaatatttatactctcagacatttcctctagatataagagatataagtttaacatcttcatttgatgatgcattattattttctaacaaagtttattcatttttttctatggattatattaaaactgctaataaagtggtagaagcaggattatttgcaggttgggtgaaacagatagtaaatgattttgtaatcgaagctaataaaagcaatactatggataaaattgcagatatatctctaattgttccttatataggattagctttaaatgtaggaaatgaaacagctaaaggaaattttgaaaatgcttttgagattgcaggagccagtattctactagaatttataccagaacttttaatacctgtagttggagcctttttattagaatcatatattgacaataaaaataaaattattaaaacaatagataatgctttaactaaaagaaatgaaaaatggagtgatatgtacggattaatagtagcgcaatggctctcaacagttaatactcaattttatacaataaaagagggaatgtataaggctttaaattatcaagcacaagcattggaagaaataataaaatacagatataatatatattctgaaaaagaaaagtcaaatattaacatcgattttaatgatataaattctaaacttaatgagggtattaaccaagctatagataatataaataattttataaatggatgttctgtatcatatttaatgaaaaaaatgattccattagctgtagaaaaattactagactttgataatactctcaaaaaaaatttgttaaattatatagatgaaaataaattatatttgattggaagtgcagaatatgaaaaatcaaaagtaaataaatacttgaaaaccattatgccgtttgatctttcaatatataccaatgatacaatactaatagaaatgtttaataaatataatagcgaaattctcgagcaccaccaccaccaccactaa

SEQ ID NO: 10 - protein, 882 aa, Clostridium botulinum
MAVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSGIITSKTKSLVPRGSKASGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILEHHHHHH

SEQ ID NO: 11 - DNA, 3885 bp, Clostridium botulinum
atggcaataacaattaacaactttaattattcagatcctgttgataataaaaatattttatatttagatactcatttaaatacattagctaatgagcctgaaaaagcctttcgcattatagggaatatatgggtaatacccgatagattttcaagagattctaatccaaatttaaataaacctccacgagttacaagccctaaaagtggttattatgatcctaattatttgagtactgattctgaaaaagatacatttttaaaagaaattataaagttatttaaaagaattaactctagagaaataggagaagaattaatatatagacttgcaacagacataccctttcctgggaataacaatactccaattaatacttttgattttgatgtagattttaacagtgttgatgttaaaactagacaaggtaacaactgggttaaaactggtagtataaatcctagtgttataataactggacctagagaaaacattatagacccagaaacttctacgtttaaattaactaacaatacttttgcggcacaagaaggatttggtgctttatcaataatttcaatatcacctagatttatgctaacatatagtaatgcaactaataatgtaggagagggtagattttctaagtctgaattttgcatggatccaatactaattttaatgcatgaacttaatcatgcaatgcataatttatatggaatagctataccaaatgatcaaagaatttcatctgtaactagtaatattttttattctcaatataatgtgaaattagagtatgcagaaatatatgcatttggaggtccaactatagaccttattcctaaaagtgcaaggaaatattttgaggaaaaggcattggattattatagatccatagctaaaagacttaatagtataactactgcaaatccttcaagctttaataaatatataggagaatataaacagaaacttattagaaagtatagattcgtagtagaatcttcaggtgaagttgcagtagatcgtaataagtttgctgagttatataaagaacttacacaaatatttacagaatttaactacgctaaaatatataatgtacaaaataggaaaatatatctttcaaatgtatatactccggttacggcaaatatattagacgataatgtttatgatatacaaaatggatttaacatacctaaaagtaatttaaatgtactatttatgggtcaaaatttatctcgaaatccagcattaagaaaagtcaatcctgaaaatatgctttatttatttacaaaattttgtcatagagggataataacttctaaaactaaatcattagttccgcgtggatccaaggccttagattgtagagagcttttagttaaaaatactgacttaccctttataggtgatattagtgatatcaaaactgatatatttttaagcaaagatattaatgaagaaactgaagttatagactatccggacaatgtttcagtggatcaagttattctcagtaagaatacctcagaacatggacaactagatttattataccctattattgaaggtgagagtcaagtattaccgggagagaatcaagtcttttatgataatagaactcaaaatgttgattatttgaattcttattattacctagaatctcaaaaactaagtgataatgttgaagattttacttttacgacatcaattgaggaagctttggataatagtggaaaagtatatacttactttcctaaactagctgataaagtaaatacgggtgttcaaggtggtttatttttaatgtgggcaaatgatgtagttgaagattttactacaaatattctaagaaaagatacattagataaaatatcagatgtatcagctattattccctatataggacctgcattaaatataagtaattctgtaagaaggggaaattttactgaagcatttgcagttaccggtgtaactattttattagaagcgtttcaagaatttacaatacctgcacttggtgcatttgtgatttatagtaaggttcaagaaagaaacgagattattaaaactatagataattgtttagaacaaaggattaaaagatggaaagattcatatgaatggatgataggaacgtggttatccaggattactactcaatttaataatataagttatcaaatgtatgattctttaaattatcaggcagatgcaatcaaagataaaatagatttagaatataaaaaatactcaggaagtgataaagaaaatataaaaagtcaagttgaaaatttaaaaaatagtttagatataaaaatctcggaagcaatgaataatataaataaatttatacgagaatgttctgtaacatacttatttaaaaatatgctccctaaagtaattgatgaattaaataagtttgatttaaaaactaaaacagaattaattaatcttatagatagtcataatattattctagttggtgaagtagatagattaaaagcaaaaataaatgagagttttgaaaatacaataccctttaatattttttcatatactaataattctttattaaaagatatactcgaggaatatttcaatagtattaatgattcaaaaattttgagcttacaaaacaaaaaaaatgctttagtggatacatcaggatataatgcagaagtgaggctagaaggtgatgttcaagttaatacgatatatacaaatgattttaaattaagtagttcaggagataaaattatagtaaatttaaataataatattttatatagcgctatttatgagaactctagtgttagtttttggattaagatatctaaagatttaactaattctcataatgaatatactataattaatagtataaaacaaaattctgggtggaaattatgtattagaaatggcaatatagaatggattttacaagatattaatagaaagtataaaagtttaatttttgattatagtgaatcattaagtcatacaggatatacaaataaatggttttttgttactataactaataatataatggggtatatgaaactttatataaatggagaattaaagcagagtgaaagaattgaagatttagatgaggttaagttagataaaaccatagtatttggaatagatgagaatatagatgagaatcagatgctttggattagagattttaatattttttctaaagaattaagcaatgaagatattaatattgtatatgagggacaaatattaagaaatgttattaaagattattggggaaatcctttgaagtttgatacagaatattatatgattaattataattatatagataggtatatagcacctaaaaataatatacttgtacttgttcagtattcagatatatctaaattatatactaaaaatcctattactattaaatcagcagctaataagaatccttatagtagaattttaaatggagatgatataatgtttcatatgttatatgatagtagggaatatatgataataagagatactgatacaatatatgcaacacaaggaggacagtgttcaaaaaattgtgtatatgcattaaaattacagagtaatttaggtaattatggtataggtatatttagtataaaaaatattgtatctcaaaataaatattgtagtcaaattttctctagttttatgaaaaatacaatgcttctagcagatatatataaaccttggagattttcttttgaaaatgcatacacgccagttgcagtaactaattatgagacaaaactattatcaacttcatctttttggaaatttatttctagggatccaggatgggtagagctcgagcaccaccaccaccaccactaa

SEQ ID NO: 12 - protein, 1294 aa, Clostridium botulinum
MAITINNFNYSDPVDNKNILYLDTHLNTLANEPEKAFRIIGNIWVIPDRFSRDSNPNLNKPPRVTSPKSGYYDPNYLSTDSEKDTFLKEIIKLFKRINSREIGEELIYRLATDIPFPGNNNTPINTFDFDVDFNSVDVKTRQGNNWVKTGSINPSVIITGPRENIIDPETSTFKLTNNTFAAQEGFGALSIISISPRFMLTYSNATNNVGEGRFSKSEFCMDPILILMHELNHAMHNLYGIAIPNDQRISSVTSNIFYSQYNVKLEYAEIYAFGGPTIDLIPKSARKYFEEKALDYYRSIAKRLNSITTANPSSFNKYIGEYKQKLIRKYRFVVESSGEVAVDRNKFAELYKELTQIFTEFNYAKIYNVQNRKIYLSNVYTPVTANILDDNVYDIQNGFNIPKSNLNVLFMGQNLSRNPALRKVNPENMLYLFTKFCHRGIITSKTKSLVPRGSKALDCRELLVKNTDLPFIGDISDIKTDIFLSKDINEETEVIDYPDNVSVDQVILSKNTSEHGQLDLLYPIIEGESQVLPGENQVFYDNRTQNVDYLNSYYYLESQKLSDNVEDFTFTTSIEEALDNSGKVYTYFPKLADKVNTGVQGGLFLMWANDVVEDFTTNILRKDTLDKISDVSAIIPYIGPALNISNSVRRGNFTEAFAVTGVTILLEAFQEFTIPALGAFVIYSKVQERNEIIKTIDNCLEQRIKRWKDSYEWMIGTWLSRITTQFNNISYQMYDSLNYQADAIKDKIDLEYKKYSGSDKENIKSQVENLKNSLDIKISEAMNNINKFIRECSVTYLFKNMLPKVIDELNKFDLKTKTELINLIDSHNIILVGEVDRLKAKINESFENTIPFNIFSYTNNSLLKDILEEYFNSINDSKILSLQNKKNALVDTSGYNAEVRLEGDVQVNTIYTNDFKLSSSGDKIIVNLNNNILYSAIYENSSVSFWIKISKDLTNSHNEYTIINSIKQNSGWKLCIRNGNIEWILQDINRKYKSLIFDYSESLSHTGYTNKWFFVTITNNIMGYMKLYINGELKQSERIEDLDEVKLDKTIVFGIDENIDENQMLWIRDFNIFSKELSNEDINIVYEGQILRNVIKDYWGNPLKFDTEYYMINYNYIDRYIAPKNNILVLVQYSDISKLYTKNPITIKSAANKNPYSRILNGDDIMFHMLYDSREYMIIRDTDTIYATQGGQCSKNCVYALKLQSNLGNYGIGIFSIKNIVSQNKYCSQIFSSFMKNTMLLADIYKPWRFSFENAYTPVAVTNYETKLLSTSSFWKFISRDPGWVELEHHHHHH

SEQ ID NO: 13 - DNA, 2625 bp, Clostridium botulinum
atggcaataacaattaacaactttaattattcagatcctgttgataataaaaatattttatatttagatactcatttaaatacattagctaatgagcctgaaaaagcctttcgcattatagggaatatatgggtaatacccgatagattttcaagagattctaatccaaatttaaataaacctccacgagttacaagccctaaaagtggttattatgatcctaattatttgagtactgattctgaaaaagatacatttttaaaagaaattataaagttatttaaaagaattaactctagagaaataggagaagaattaatatatagacttgcaacagacataccctttcctgggaataacaatactccaattaatacttttgattttgatgtagattttaacagtgttgatgttaaaactagacaaggtaacaactgggttaaaactggtagtataaatcctagtgttataataactggacctagagaaaacattatagacccagaaacttctacgtttaaattaactaacaatacttttgcggcacaagaaggatttggtgctttatcaataatttcaatatcacctagatttatgctaacatatagtaatgcaactaataatgtaggagagggtagattttctaagtctgaattttgcatggatccaatactaattttaatgcatgaacttaatcatgcaatgcataatttatatggaatagctataccaaatgatcaaagaatttcatctgtaactagtaatattttttattctcaatataatgtgaaattagagtatgcagaaatatatgcatttggaggtccaactatagaccttattcctaaaagtgcaaggaaatattttgaggaaaaggcattggattattatagatccatagctaaaagacttaatagtataactactgcaaatccttcaagctttaataaatatataggagaatataaacagaaacttattagaaagtatagattcgtagtagaatcttcaggtgaagttgcagtagatcgtaataagtttgctgagttatataaagaacttacacaaatatttacagaatttaactacgctaaaatatataatgtacaaaataggaaaatatatctttcaaatgtatatactccggttacggcaaatatattagacgataatgtttatgatatacaaaatggatttaacatacctaaaagtaatttaaatgtactatttatgggtcaaaatttatctcgaaatccagcattaagaaaagtcaatcctgaaaatatgctttatttatttacaaaattttgtcatagagggataataacttctaaaactaaatcattagttccgcgtggatccaaggccttagattgtagagagcttttagttaaaaatactgacttaccctttataggtgatattagtgatatcaaaactgatatatttttaagcaaagatattaatgaagaaactgaagttatagactatccggacaatgtttcagtggatcaagttattctcagtaagaatacctcagaacatggacaactagatttattataccctattattgaaggtgagagtcaagtattaccgggagagaatcaagtcttttatgataatagaactcaaaatgttgattatttgaattcttattattacctagaatctcaaaaactaagtgataatgttgaagattttacttttacgacatcaattgaggaagctttggataatagtggaaaagtatatacttactttcctaaactagctgataaagtaaatacgggtgttcaaggtggtttatttttaatgtgggcaaatgatgtagttgaagattttactacaaatattctaagaaaagatacattagataaaatatcagatgtatcagctattattccctatataggacctgcattaaatataagtaattctgtaagaaggggaaattttactgaagcatttgcagttaccggtgtaactattttattagaagcgtttcaagaatttacaatacctgcacttggtgcatttgtgatttatagtaaggttcaagaaagaaacgagattattaaaactatagataattgtttagaacaaaggattaaaagatggaaagattcatatgaatggatgataggaacgtggttatccaggattactactcaatttaataatataagttatcaaatgtatgattctttaaattatcaggcagatgcaatcaaagataaaatagatttagaatataaaaaatactcaggaagtgataaagaaaatataaaaagtcaagttgaaaatttaaaaaatagtttagatataaaaatctcggaagcaatgaataatataaataaatttatacgagaatgttctgtaacatacttatttaaaaatatgctccctaaagtaattgatgaattaaataagtttgatttaaaaactaaaacagaattaattaatcttatagatagtcataatattattctagttggtgaagtagatagattaaaagcaaaaataaatgagagttttgaaaatacaataccctttaatattttttcatatactaataattctttattaaaagatatactcgagcaccaccaccaccaccactaa

SEQ ID NO: 14 - protein, 874 aa, Clostridium botulinum
MAITINNFNYSDPVDNKNILYLDTHLNTLANEPEKAFRIIGNIWVIPDRFSRDSNPNLNKPPRVTSPKSGYYDPNYLSTDSEKDTFLKEIIKLFKRINSREIGEELIYRLATDIPFPGNNNTPINTFDFDVDFNSVDVKTRQGNNWVKTGSINPSVIITGPRENIIDPETSTFKLTNNTFAAQEGFGALSIISISPRFMLTYSNATNNVGEGRFSKSEFCMDPILILMHELNHAMHNLYGIAIPNDQRISSVTSNIFYSQYNVKLEYAEIYAFGGPTIDLIPKSARKYFEEKALDYYRSIAKRLNSITTANPSSFNKYIGEYKQKLIRKYRFVVESSGEVAVDRNKFAELYKELTQIFTEFNYAKIYNVQNRKIYLSNVYTPVTANILDDNVYDIQNGFNIPKSNLNVLFMGQNLSRNPALRKVNPENMLYLFTKFCHRGIITSKTKSLVPRGSKALDCRELLVKNTDLPFIGDISDIKTDIFLSKDINEETEVIDYPDNVSVDQVILSKNTSEHGQLDLLYPIIEGESQVLPGENQVFYDNRTQNVDYLNSYYYLESQKLSDNVEDFTFTTSIEEALDNSGKVYTYFPKLADKVNTGVQGGLFLMWANDVVEDFTTNILRKDTLDKISDVSAIIPYIGPALNISNSVRRGNFTEAFAVTGVTILLEAFQEFTIPALGAFVIYSKVQERNEIIKTIDNCLEQRIKRWKDSYEWMIGTWLSRITTQFNNISYQMYDSLNYQADAIKDKIDLEYKKYSGSDKENIKSQVENLKNSLDIKISEAMNNINKFIRECSVTYLFKNMLPKVIDELNKFDLKTKTELINLIDSHNIILVGEVDRLKAKINESFENTIPFNIFSYTNNSLLKDILEHHHHHH

SEQ ID NO: 15 - DNA, 3810 bp, Clostridium botulinum
atggcaacaattaatagttttaattataatgatcctgttaataatagaacaattttatatattaaaccaggcggttgtcaacaattttataaatcatttaatattatgaaaaatatttggataattccagagagaaatgtaattggtacaattccccaagattttcttccgcctacttcattgaaaaatggagatagtagttattatgaccctaattatttacaaagtgatcaagaaaaggataaatttttaaaaatagtcacaaaaatatttaatagaataaatgataatctttcaggaaggattttattagaagaactgtcaaaagctaatccatatttaggaaatgataatactccagatggtgacttcattattaatgatgcatcagcagttccaattcaattctcaaatggtagccaaagcatactattacctaatgttattataatgggagcagagcctgatttatttgaaactaacagttccaatatttctctaagaaataattatatgccaagcaatcacggttttggatcaatagctatagtaacattctcacctgaatattcttttagatttaaagataatagtatgaatgaatttattcaagatcctgctcttacattaatgcatgaattaatacattcattacatggactatatggggctaaagggattactacaaagtatactataacacaaaaacaaaatcccctaataacaaatataagaggtacaaatattgaagaattcttaacttttggaggtactgatttaaacattattactagtgctcagtccaatgatatctatactaatcttctagctgattataaaaaaatagcgtctaaacttagcaaagtacaagtatctaatccactacttaatccttataaagatgtttttgaagcaaagtatggattagataaagatgctagcggaatttattcggtaaatataaacaaatttaatgatatttttaaaaaattatacagctttacggaatttgatttagcaactaaatttcaagttaaatgtaggcaaacttatattggacagtataaatacttcaaactttcaaacttgttaaatgattctatttataatatatcagaaggctataatataaataatttaaaggtaaattttagaggacagaatgcaaatttaaatcctagaattattacaccaattacaggtagaggactagtaaaaaaaatcattagattttgtaaaagtgggataataacttctaaaactaaatcattggttccacgtggatccaaggcctcaatatgtatcgaaataaataatggtgagttattttttgtggcttccgagaatagttataatgatgataatataaatactcctaaagaaattgacgatacagtaacttcaaataataattatgaaaatgatttagatcaggttattttaaattttaatagtgaatcagcacctggactttcagatgaaaaattaaatttaactatccaaaatgatgcttatataccaaaatatgattctaatggaacaagtgatatagaacaacatgatgttaatgaacttaatgtatttttctatttagatgcacagaaagtgcccgaaggtgaaaataatgtcaatctcacctcttcaattgatacagcattattagaacaacctaaaatatatacatttttttcatcagaatttattaataatgtcaataaacctgtgcaagcagcattatttgtaggctggatacaacaagtattagtagattttactactgaagctaaccaaaaaagtactgttgataaaattgcagatatttctatagttgttccatatataggtcttgctttaaatataggaaatgaagcacaaaaaggaaattttaaagatgcacttgaattattaggagcaggtattttattagaatttgaacccgagcttttaattcctacaattttagtattcacgataaaatcttttttaggttcatctgataataaaaataaagttattaaagcaataaataatgcattgaaagaaagagatgaaaaatggaaagaagtatatagttttatagtatcgaattggatgactaaaattaatacacaatttaataaaagaaaagaacaaatgtatcaagctttacaaaatcaagtaaatgcacttaaagcaataatagaatctaagtataatagttatactttagaagaaaaaaatgagcttacaaataaatatgatattgagcaaatagaaaatgaacttaatcaaaaggtttctatagcaatgaataatatagacaggttcttaactgaaagttctatatcttatttaatgaaattaataaatgaagtaaaaattaataaattaagagaatatgatgaaaatgttaaaacgtatttattagattatattataaaacatggatcaatcttgggagagagtcagcaagaactaaattctatggtaattgataccctaaataatagtattccttttaagctttcttcttatacagatgataaaattttaatttcatattttaataagttctttaagagaattaaaagtagttctgttttaaatatgagatataaaaatgataaatacgtagatacttcaggatatgattcaaatataaatattaatggagatgtatataaatatccaactaataaaaatcaatttggaatatataatgataaacttagtgaagttaatatatctcaaaatgattacattatatatgataataaatataaaaattttagtattagtttttgggtaagaattcctaactatgataataagatagtaaatgttaataatgaatacactataataaattgtatgagggataataattcaggatggaaagtatctcttaatcataatgaaataatttggacattgcaagataattcaggaattaatcaaaaattagcatttaactatggtaacgcaaatggtatttctgattatataaataagtggatttttgtaactataactaatgatagattaggagattctaaactttatattaatggaaatttaatagataaaaaatcaattttaaatttaggtaatattcatgttagtgacaatatattatttaaaatagttaattgtagttatacaagatatattggtattagatattttaatatttttgataaagaattagatgaaacagaaattcaaactttatataacaatgagcctaatgcaaatattttaaaggatttttggggaaattatttgctttatgacaaagaatattatttattaaatgtgttaaaaccaaataactttattaataggagaacagattctactttaagcattaataatataagaagcactattcttttagctaatagattatatagtggaataaaagttaaaatacaaagagttaataatagtagtactaacgataatcttgttagaaagaatgatcaggtatatattaattttgtagccagcaaaactcacttacttccattatatgctgatacagctaccacaaataaagagaaaacaataaaaatatcatcatctggcaatagatttaatcaagtagtagttatgaattcagtaggaaataattgtacaatgaattttaaaaataataatggaaataatattgggttgttaggtttcaaggcagatactgtagttgctagtacttggtattatacacatatgagagataatacaaacagcaatggatttttttggaactttatttctgaagaacatggatggcaagaaaaaacgcgttctcaccaccaccaccaccactaa

SEQ ID NO: 16 - protein, 1269 aa, Clostridium botulinum
MATINSFNYNDPVNNRTILYIKPGGCQQFYKSFNIMKNIWIIPERNVIGTIPQDFLPPTSLKNGDSSYYDPNYLQSDQEKDKFLKIVTKIFNRINDNLSGRILLEELSKANPYLGNDNTPDGDFIINDASAVPIQFSNGSQSILLPNVIIMGAEPDLFETNSSNISLRNNYMPSNHGFGSIAIVTFSPEYSFRFKDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKSGIITSKTKSLVPRGSKASICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVGWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNALKAIIESKYNSYTLEEKNELTNKYDIEQIENELNQKVSIAMNNIDRFLTESSISYLMKLINEVKINKLREYDENVKTYLLDYIIKHGSILGESQQELNSMVIDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNSGINQKLAFNYGNANGISDYINKWIFVTITNDRLGDSKLYINGNLIDKKSILNLGNIHVSDNILFKIVNCSYTRYIGIRYFNIFDKELDETEIQTLYNNEPNANILKDFWGNYLLYDKEYYLLNVLKPNNFINRRTDSTLSINNIRSTILLANRLYSGIKVKIQRVNNSSTNDNLVRKNDQVYINFVASKTHLLPLYADTATTNKEKTIKISSSGNRFNQVVVMNSVGNNCTMNFKNNNGNNIGLLGFKADTVVASTWYYTHMRDNTNSNGFFWNFISEEHGWQEKTRSHHHHHH

SEQ ID NO: 17 - DNA, 1116 bp, Pseudomonas aeruginosa
atgggcagcctggccgcgctgaccgcgcaccaggcttgccacagagggataataacttctaaaactaaatcattagttccgcgtggatccaaggccctggagcagtgcgggtacccggtgcagcggctggtcgccctctacctggcggcgcggctgtcgtggaaccaggtcgaccaggtgatccgcaacgccctggccagccccggcagcggcggcgacctgggcgaagcgatccgcgagcagccggagcaggcccgtctggccctgaccctggccgccgccgagagcgagcgcttcgtccggcagggcaccggcaacgacgaggccggcgcggccaacgccgacgtggtgagcctgacctgcccggtcgccgccggtgaatgcgcgggcccggcggacagcggcgacgccctgctggagcgcaactatcccactggcgcggagttcctcggcgacggcggcgacgtcagcttcagcacccgcggcacgcagaactggacggtggagcggctgctccaggcgcaccgccaactggaggagcgcggctatgtgttcgtcggctaccacggcaccttcctcgaagcggcgcaaagcatcgtcttcggcggggtgcgcgcgcgcagccaggacctcgacgcgatctggcgcggtttctatatcgccggcgatccggcgctggcctacggctacgcccaggaccaggaacccgacgcacgcggccggatccgcaacggtgccctgctgcgggtctatgtgccgcgctcgagcctgccgggcttctaccgcaccagcctgaccctggccgcgccggaggcggcgggcgaggtcgaacggctgatcggccatccgctgccgctgcgcctggacgccatcaccggccccgaggaggaaggcgggcgcctggagaccattctcggctggccgctggccgagcgcaccgtggtgattccctcggcgatccccaccgacccgcgcaacgtcggcggcgacctcgacccgtccagcatccccgacaaggaacaggcgatcagcgccctgccggactacgccagccagcccggcaaaccgccgcgcgaggacctgacgcgtctcgagcaccaccaccaccaccactaa

SEQ ID NO: 18 - protein, 371 aa, Pseudomonas aeruginosa
MGSLAALTAHQACHRGIITSKTKSLVPRGSKALEQCGYPVQRLVALYLAARLSWNQVDQVIRNALASPGSGGDLGEAIREQPEQARLALTLAAAESERFVRQGTGNDEAGAANADVVSLTCPVAAGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPREDLTRLEHHHHHH

SEQ ID NO: 19 - DNA, 1215 bp, Corynebacterium diphtheriae
atggctgatgatgttgttgattcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataacgaaaacccgctctctggaaaagctggaggcgtggtcaaagtgacgtatccaggactgacgaaggtcctcgcactaaaagtggataatgccgaaactattaagaaagagttaggtttaagtctcactgaaccgttgatggagcaagtcggaacagaagagtttatcaaaaggttcggggatggtgcttcgcgtgtagtactcagccttccctttgccgaggggagttctagcgtcgaatatattaataactgggaacaggcgaaagcgttaagcgtagaacttgagattaattttgaaacccgtggaaaacgtggccaagatgcgatgtatgagtatatggctcaagcctgtgcaagagggataataacttctaaaactaaatcattggttccacgtggatccaaggccttgtcatgcataaatcttgattgggatgtcataagggataaaactaagacaaagatagagtctttgaaagagcatggccctatcaaaaataaaatgagcgaaagtcccaataaaacagtatctgaggaaaaagctaaacaatacctagaagaatttcatcaaaccgcattagagcatcctgaattgtcagaacttaaaaccgttactgggaccaatcctcttttcgctggggctaactatgcggcgtgggcagtaaacgttgcgcaagttatcgatagcgaaacagctgataatttggaaaagacaactgctgctctttcgatacttcctggtatcggtagcgtaatgggcattgcagacggtgccgttcaccacaatacagaagagatagtggcacaatcaatagctttatcgtctttaatggttgctcaagctattccattggtaggagagctagttgatattggtttcgctgcatataattttgtagagagtattatcaatttgtttcaagtagttcataattcgtataatcgtcccgcgtattctccggggcataaaacgcaaccatttctcgagcaccaccaccaccaccactaa

SEQ ID NO: 20 - protein, 404 aa, Corynebacterium diphtheriae
MADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACARGIITSKTKSLVPRGSKALSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLEHHHHHH

SEQ ID NO: 21 - DNA, 1650 bp, Ricinus communis
atggtccccaaacaatacccaattataaactttaccacagcgggtgccactgtgcaaagctacacaaactttatcagagctgttcgcggtcgtttaacaactggagctgatgtgagacatgatataccagtgttgccaaacagagttggtttgcctataaaccaacggtttattttagttgaactctcaaatcatgcagagctttctgttacattagccctggatgtcaccaatgcatatgtggtcggctaccgtgctggaaatagcgcatatttctttcatcctgacaatcaggaagatgcagaagcaatcactcatcttttcactgatgttcaaaatcgatatacattcgcctttggtggtaattatgatagacttgaacaacttgctggtaatctgagagaaaatatcgagttgggaaatggtccactagaggaggctatctcagcgctttattattacagtactggtggcactcagcttccaactctggctcgttcctttataatttgcatccaaatgatttcagaagcagcaagattccaatatattgagggagaaatgcgcacgagaattaggtacaaccggagatctgcaccagatcctagcgtaattacacttgagaatagttgggggagactttccactgcaattcaagagtctaaccaaggagcctttgctagtccaattcaactgcaaagacgtaatggttccaaattcagtgtgtacgatgtgagtatattaatccctatcatagctctcatggtgtatagatgcgcacctcctagggggataataacttctaaaactaaatcattggttccacgtggatccaaggctgatgtttgtatggatcctgagcccatagtgcgtatcgtaggtcgaaatggtctatgtgttgatgttagggatggaagattccacaacggaaacgcaatacagttgtggccatgcaagtctaatacagatgcaaatcagctctggactttgaaaagagacaatactattcgatctaatggaaagtgtttaactacttacgggtacagtccgggagtctatgtgatgatctatgattgcaatactgctgcaactgatgccacccgctggcaaatatgggataatggaaccatcataaatcccagatctagtctagttttagcagcgacatcagggaacagtggtaccacacttacggtgcaaaccaacatttatgccgttagtcaaggttggcttcctactaataatacacaaccttttgttacaaccattgttgggctatatggtctgtgcttgcaagcaaatagtggacaagtatggatagaggactgtagcagtgaaaaggctgaacaacagtgggctctttatgcagatggttcaatacgtcctcagcaaaaccgagataattgccttacaagtgattctaatatacgggaaacagttgttaagatcctctcttgtggccctgcatcctctggccaacgatggatgttcaagaatgatggaaccattttaaatttgtatagtggattggtgttagatgtgaggcgatcggatccgagccttaaacaaatcattctttaccctctccatggtgacccaaaccaaatatggttaccattatttctcgagcaccaccaccaccaccactaa

SEQ ID NO: 22 - protein, 549 aa, Ricinus communis
MVPKQYPIINFTTAGATVQSYTNFIRAVRGRLTTGADVRHDIPVLPNRVGLPINQRFILVELSNHAELSVTLALDVTNAYVVGYRAGNSAYFFHPDNQEDAEAITHLFTDVQNRYTFAFGGNYDRLEQLAGNLRENIELGNGPLEEAISALYYYSTGGTQLPTLARSFIICIQMISEAARFQYIEGEMRTRIRYNRRSAPDPSVITLENSWGRLSTAIQESNQGAFASPIQLQRRNGSKFSVYDVSILIPIIALMVYRCAPPRGIITSKTKSLVPRGSKADVCMDPEPIVRIVGRNGLCVDVRDGRFHNGNAIQLWPCKSNTDANQLWTLKRDNTIRSNGKCLTTYGYSPGVYVMIYDCNTAATDATRWQIWDNGTIINPRSSLVLAATSGNSGTTLTVQTNIYAVSQGWLPTNNTQPFVTTIVGLYGLCLQANSGQVWIEDCSSEKAEQQWALYADGSIRPQQNRDNCLTSDSNIRETVVKILSCGPASSGQRWMFKNDGTILNLYSGLVLDVRRSDPSLKQIILYPLHGDPNQIWLPLFLEHHHHHH

(19) United States
(12) Patent Application Publication
(10) Pub. No.: US 2008/0103098 A1
(43) Pub. Date: May 1, 2008

(54) Recombinant Expression of Proteins in a Disulfide-Bridged, Two-Chain Form

(75) Inventor: Volker Specht, Berlin (DE)

Correspondence Address: GUDRUN E. HUCKETT DRAUDT, SCHUBERTSTR. 15A, WUPPERTAL, 42289, DE

(73) Assignee: BIOTECON THERAPEUTICS GMBH, Potsdam (DE)

(21) Appl. No.: 11/814,511

(22) PCT Filed: Jan. 20, 2006
(86) PCT No.: PCT/DE06/00088

§ 371 Date: Jul. 23, 2007

(30) Foreign Application Priority Data
Jan. 21, 2005 (DE) 10 2005 002 978.7

(51) Int. Cl. A61K 38/00; A61P 43/00; C07H 21/04; C07K 16/00; C12N 15/00; C12N 5/02; C12P 21/04

(52) U.S. Cl. 514/12; 435/252.33; 435/320.1; 435/69.1; 530/350; 536/23.7

(57) ABSTRACT

Polypeptides or proteins are produced as a disulfide bridged dichain by recombinant expression in E. coli host cells and exert biologic activity as such a dichain. A C-terminal amino acid of the first chain is Arg or Lys. The second chain has N-terminally 1 to 20 amino acid residues and a PRS sequence VPXGS, wherein X is a natural amino acid; V is Val, Leu, Ile, Ala, Phe, Pro or Gly; P is Pro, Leu, Ile, Ala, Phe, Val or Gly; G is Gly, Leu, Ile, Ala, Pro, Phe or Val; S is Ser, Tyr, Trp, or Thr. The polypeptide/protein is modified at the nucleic acid level to a construct that in a loop area has a PRS sequence VPXGS with X, V, P, G, and S as defined. The construct is inserted into E. coli host cells that are cultivated and subsequently lysed for isolating the dichain disulfide-bridged peptide/protein.

One aspect of the present invention concerns a method for producing proteins in a dichain form by means of recombinant expression in E. coli host cells. Another aspect of the present invention concerns proteins or polypeptides in dichain and biologically active form that can be produced by means of the aforementioned method.

The important advantage in comparison to corresponding recombinant proteins/polypeptides that do not exhibit the features according to the invention resides in that they must not be treated with a specific protease for targeted cleavage of the polypeptide chain so that the method of production is significantly simplified. Further aspects of the present invention are nucleic acids that code for the polypeptides/protein according to the present invention; vectors that contain such nucleic acids or nucleic acid sequences; host cells that, in turn, contain the aforementioned vectors; and, finally, pharmaceutical preparations that contain the dichain and biologically active proteins/polypeptides.

Clostridial neurotoxins are strong inhibitors of the calcium-dependent neurotransmitter secretion in neuronal cells. After oral uptake of botulinum toxins (BoNT), for example, through spoiled foods, a clinical picture referred to as botulism that is characterized by paralysis of various muscles will show. Paralysis of the breathing muscles can finally lead to the death of the affected person. In this connection, the signal transfer from the nerve to the muscle is interrupted at the myoceptor because the motor neurons can no longer excrete acetyl choline. The botulinum neurotoxins develop their inhibiting action by means of the proteolytic cleavage of the proteins participating in the secretion processes, the so-called SNARE proteins. In this context, the neurotoxins of different serotypes have different specificity with regard to the SNARE proteins and the cleavage sites at the respective amino acid sequences. BoNT(A) and BoNT(E) cleave the SNARE protein SNAP-25 while BoNT(C) recognizes SNAP-25 as well as syntaxin-1 as a substrate. Also, the toxins of the serotypes B, D, F, and G as well as the tetanus toxin (TeNT) cleave VAMP-2 (synaprobrevin-2) (Schiavo et al., 1997).

The clostridial neurotoxins are the strongest known poisons. For example, the intravenously administered lethal dose at which half of all mice of a dosage group will die of botulism is only 5 pg. That the toxins of most serotypes are toxic also when orally administered is the result of complex proteins in which they are embedded and which therefore protect them from being decomposed by digestive enzymes as they pass through the gastrointestinal tract. They also are attributed a function in resorption of the toxins through the small intestine epithelium (Fujinaga, 1997).

During the past decades, the botulinum toxins of the serotypes A and B have found therapeutic uses. For example, it is possible by a targeted injection of only minimal doses to relax individual chronically cramped muscles. A particular advantage is the long effectiveness of, for example, BoNT(A) and BoNT(B) for more than three to six months. First indications have been, inter alia, dystonia such as torticollia, blepharospasm, and strabism; additional ones such as hyperhidrosis or cosmetic treatments for smoothing wrinkles have been added. The market for botulinum toxin as a therapeutic agent grows rapidly, not least because of the development of further indications and the more intensive utilization in already existing applications. In this connection, there are attempts to improve the properties of the neurotoxins with regard to duration of activity, potency, and the antigen potential. Tests have shown that the complex proteins that are contained in the commercially available preparations (BOTOX available from Allergen and Dysport available from Ipsen-Beaufort as BoNT(A) preparations as well as Myobloc/Neurobloc available from Elan as BoNT(B) preparation) have no positive effects on the duration of activity and the potency, but, because of the higher protein quantity in comparison to a preparation of the pure neurotoxin with the same activity, can cause the triggering of immunoreactions in the patient so that further injections become ineffective.

Since the complex proteins are not required in the active ingredient formulation and are even disadvantageous and some modifications for improvement of the properties can be achieved only by gene technology, there is a great need to produce the neurotoxins by recombinant expression, for example, by expression in Escherichia coli (neurotoxins generated in this way are free of the aforementioned complex proteins). New indications are to be developed moreover in that the botulinum toxins are to be imparted with a different cell specificity. In this connection, the path via a recombinant toxin or toxin derivative is also preferred.

The botulinum toxins as well as the tetanus toxin have high homologies with regard to their amino acid sequence and are similar in particular in regard to their domain structure. They are comprised of a receptor binding domain (H_{C}), a translocation domain (H_{N}), and a catalytic subunit (L) that effects in the nerve cell the cleavage of the corresponding SNARE protein. H_{C} is responsible for the specific binding of the neurotoxins to the myoceptors while the translocation domain ensures that L can pass from the endosomes into the cytoplasm of the neurons. H_{N} (N-terminal end) and H_{C} (C-terminal end) form the heavy chain of 100 kDa while L is the light chain and forms the catalytic subunit of 50 kDa. Both polypeptide chains are connected to one another by a disulfide bridge. Between the participating cysteine residues, a linker area or loop area (synonymously also referred to as linker sequence or loop sequence or, simpler, as linker or loop) whose length between the botulinum toxins of the individual serotypes varies greatly. At the latest at the time of release of the toxins from the clostridia during the course of cell lysis, the loop is cleaved by a clostridial endopeptidase that has not been characteristic until now wherein the ratio of cleaved and uncleaved species between the serotypes varies. For the activity of the neurotoxins the cleavage of the loop to the dichain toxin is essential (Schiavo et al., 1997). For example, in the case of the botulinum neurotoxin A a decapeptide is cut from the loop, i.e., in the loop sequence VRGIITSKTKSLDKGYNKALNDL, that has at the N-terminal end as well as at the C-terminal end a cysteine residue as an immediate neighbor, not only one peptide bond is cleaved but two proteolytic cleaving actions occurs. In this connection, the molecular weight of the biologically active botulinum neurotoxin A is naturally below that of the original clostridially translated toxin.

Since the clostridial protease is not present in other host organisms such as Escherichia coli recombinant botulinum toxins and their fragments or derivatives are expressed as single-chain peptides therein. This holds true likewise also for any other proteins that exert their normal biologic/biochemical activity as a dichain protein: In general, such proteins are obtained by means of recombinant DNA technology as single-chain proteins, their biologic/biochemical activity that they exert naturally as dichain proteins is therefore hardly present or not present at all.

In order to generate an active protein, in particular, an active botulinum toxin, the insertion of a recognition sequence for a sequence-specific protease, such as thrombin, factor Xa AA or genenase, has been necessary in the past so that, after purification, cleavage and thus activation can be performed by addition of an endoprotease. The use of such an endoprotease has essentially two disadvantages: On the one hand, it cannot always be excluded that other additional cleavage sites, in addition to the one cleavage site that has been added by gene technological measures, are present in the amino acid sequence. Even when at these secondary cleavage sites cutting is done significantly more inefficiently, after the protease treatment a mixture of different cleavage variants of the toxin can result that can be separated only with difficulty. On the other hand, in the case of pharmaceutical preparations for reasons of pharmaceutical law (regulatory considerations) it is a significant disadvantage to add subsequently a protein or to allow contact of the preparation with an additional protein because the complete removal of this protein and of its optionally existing contaminants in the further processing must be proven; this, in general, requires a significant expenditure.

An activation by proteolytic cleavage to a dichain disulfide-bridged polypeptide is required also in the case of other bacterial toxins, for example, the pseudomonas exotoxin or the diphtheria toxin in order for the enzymatic domain to exert the toxic action (for example, by ADP ribosylation of an elongation factor and thus inhibition of the protein synthesis). These toxins are employed for producing so-called immunotoxins that are used particularly in tumor therapy. For this purpose, the cell binding domain of the toxin is exchanged for a protein domain that has a high binding affinity to a tumor-specific surface protein (differentiation antigen or tumor-associated antigen). While in classic immunotoxins these protein domains are comprised of a monoclonal antibody or a fragment thereof, the specificity for certain tumor cells can also be imparted by means of cytokines, growth factors as well as mutated and selected proteins of the family of affilins, ankyrin repeat proteins, or anticalins, to name a few examples. In the recombinant expression of such fusion proteins, single-chain polypeptides are obtained. While, for example, ricin has no processing site for proteases except that of Ricinus communis and such a site must be inserted, the diphtheria toxin fragments and pseudomonas exotoxin fragments as components of the immunotoxins can be cleaved after the internalization in the endosomal compartment by a protease of the target cell. This is done in the loop area between the cysteine residues that form a disulfide bridge. However, only a minimal portion and not all internalized immunotoxin molecules are processed in this way but (Ogata et al., 1990).

In order to obtain recombinant proteins, in particular, smaller polypeptides, in sufficient quantities and in a soluble form, it is necessary in many cases to express them as a fusion protein or hybrid protein with, for example, glutathione-S-transferase or maltose binding protein in Escherichia coli. Moreover, numerous expression systems are on the market by which the desired polypeptide is expressed by means of an N-terminal or C-terminal tag for affinity purification, e.g., a His tag, Strep tag or FLAG tag. In many situations, in the expression plasmid there is a protease recognition sequence between the multiple cloning site where the DNA sequence coding for the desired protein is inserted and the coding sequence for the fusion partner or the affinity tag. This sequence is designed to enable that after expression and purification of the fusion protein the desired protein by addition of an appropriate sequence-specific endoprotease (for example, thrombin, factor Xa, or genenase) can be separated from the additional peptide areas. If the two fusion partners were bonded covalently with one another by a disulfide bridge instead of a peptide bond, a separation from one another after purification by means of a simple reduction with thiol-containing substances such as β-mercaptoethanol, DTT, or reduced glutathione would be possible. For example, the desired protein could be eluated from an affinity matrix for example, Ni-NTA agarose or StrepTactin sepharose with the aforementioned reducing agents while the affinity tag remains bonded to the matrix. A further purification step for separating the affinity tag or an added endoprotease could thus be eliminated.

It would therefore be desirable to provide a method of recombinant expression of proteins/polypeptides in general, in particular, of neurotoxins as well as fragments and derivatives of said neurotoxins and of fusion proteins or hybrid proteins, in particular, of immunotoxins that are already present after lysis of the host cells in their biological active dichain structure, wherein the two chains are disulfide-bridged. Such a method for producing such proteins and polypeptides is provided by the invention described herein.

Surprisingly, the inventor has found that the LH_{N} fragment of the BoNT(A) as well as the complete neurotoxin A, both obtained by recombinant expression as a single chain but exerting their normal biological/biochemical activity in a dichain disulfide-bridged form, are obtained by recombinant expression in a dichain form when the LH_{N} fragment or the complete toxin, preferably at the nucleic acid level, is subjected to at least one certain modification. Subsequent tests done by the inventor have shown that the same holds true also for any other proteins/polypeptides inasmuch as they are obtained in accordance with conventional recombinant methods as a single chain but exert their biological activity in a dichain disulfide-bridged form.

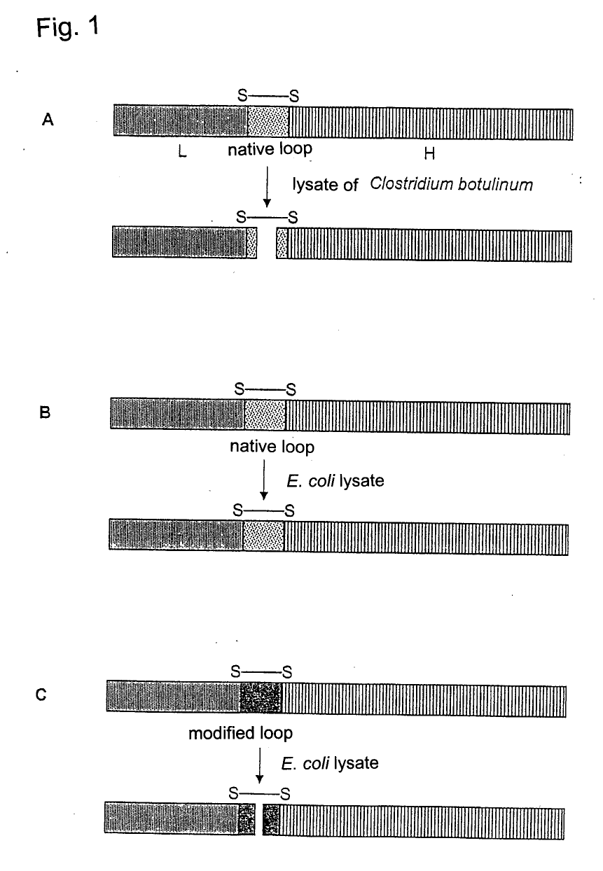

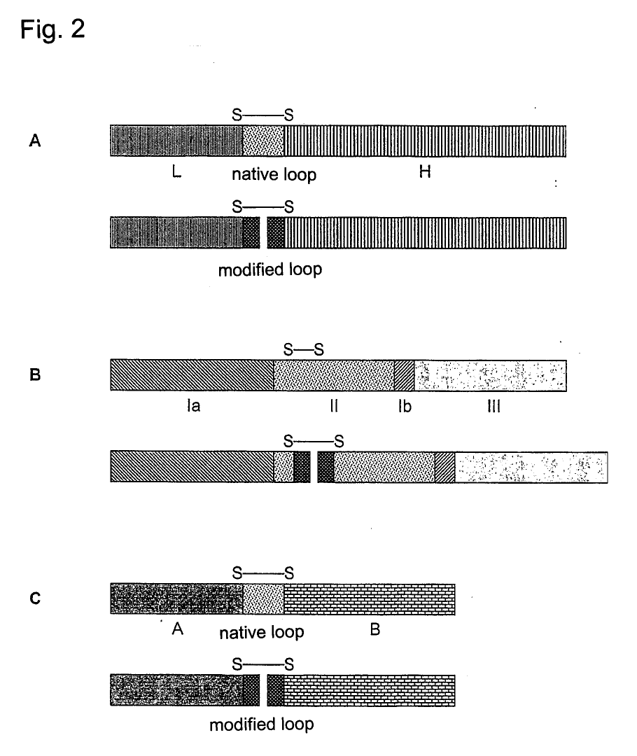

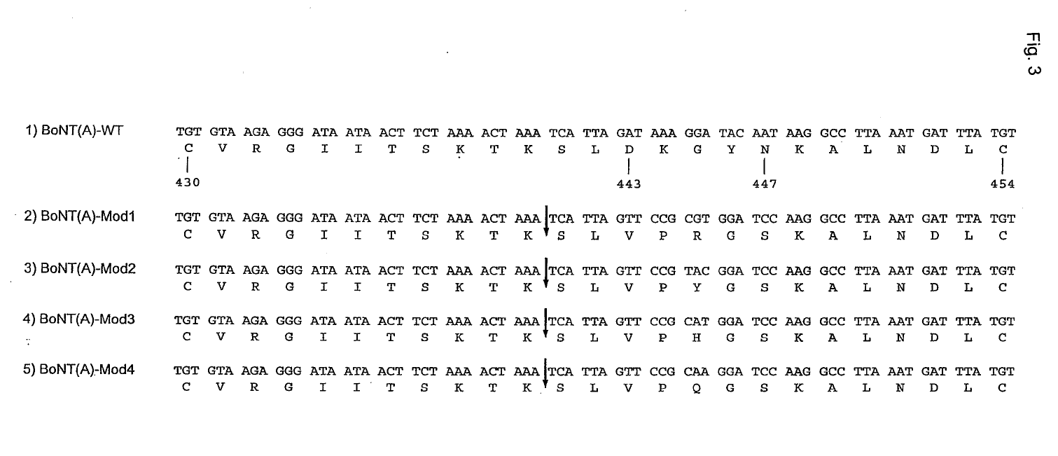

The aforementioned “at least one modification” in the case of the BoNT(A) or in the case of the LH_{N} fragment of BoNT(A) concerns the insertion of a pentapeptide sequence referred to herein as PRS (protease recognition site). In the general case of the protein/polypeptide, a pentapeptide sequence that is present in the protein/polypeptide to be modified (preferably at the nucleic acid level) can be modified in such a way (for example, by at least one exchange of an amino acid residue or by insertion of only a few amino acid residues of PRS or by deletion of amino acid residues) that it matches the pentapeptide sequence PRS inserted into the already present sequence. In the same way, a hexa/hepta/octa (etc.) peptide sequence can be inserted with or without requiring deletion of one or two or three or several amino acid residues. In accordance with the invention, it is only advantageous that the finally expressed polypeptide has the PRS (pentapeptide) sequence in its loop area wherein the loop area according to the invention is defined as the amino acid sequence that is located between the two cysteine residues participating in the disulfide bridge. When this PRS sequence is present in the loop area, this has the consequence that upon cleavage of the single-chain polypeptide adjacent to the polypeptide sequence PRS (at the amino acid level) the sequences that are naturally present in two different chains are also distributed onto two different chains. In the case of botulinum neurotoxin A (BoNT(A)), this PRS sequence is preferably inserted into the loop by deleting the pentapeptide Asp₄₄₃-Asp₄₄₇ of BoNT(A) (see FIG. 3-1). In other proteins/polypeptides (for example, in the case of BoNT(B), BoNT(C1), BoNT(D), BoNT (E), in the case of ricin, in the case of PE40 of the pseudomonas exotoxins or in the case of diphtheria toxin (DT)), it is instead preferred to insert a modified loop of BoNT(A) into the loop sequence (see FIGS. 3-2 to 3-5), wherein the amino acid residues of the natural loop sequence can be deleted or not. The modified loop sequence in FIGS. 3-2 to 3-5 are those sequences without the two terminal Cys residues wherein the central amino acid of the PRS sequence can be not only R, Y, H, or Q but also any other naturally occurring amino acid. In the case of the aforementioned other proteins/polypeptides it is particularly preferred to insert only a part of the modified loop of BoNT(A), in particular, the sequence GIITSKTKSLVPXGSKALNDL (X=a naturally occurring amino acid), wherein the amino acid residues of the natural loop sequence can be deleted or not). The modified loop sequences in FIGS. 3-2 to 3-5 are those sequences without the two terminal Cys residues.

For the LH_{N} fragment of BoNT(A) or for the complete recombinant toxin, this means thus that the sequence modification is a change in the loop area between L and H_{N} and this change provides for the presence of a PRS sequence. According to the invention, the PRS sequence, and not only for BoNT(A), is the pentapeptide sequence Val-Pro-Xaa-Gly-Ser. Xaa stands for any naturally occurring amino acid. Independent of whether Xaa is Arg or any other naturally occurring amino acid, the pentapeptide sequence Val-Pro-Xaa-Gly-Ser is referred to in any case as a pentapeptide sequence. When however one of the four other amino acid residues of the PRS sequence is exchanged, which is possible indeed within the context of the present invention, in particular, by corresponding hydrophilic/hydrophobic or polar unipolar residues, this will be referred to in this context and in the following as a variant of the PRS-pentapeptide sequence. Variants are present, for example, when Val is replaced by Leu, Ile, Ala, Phe, Pro, or Gly. Moreover, variants are present when (also or only) proline at the second position of the PRS, viewed from the N-terminal end, is replaced by Leu, Ile, Ala, Phe, Val, or Gly. Also, glycine at the fourth position of the PRS can be, for example, replaced by Leu, Ile, Ala, Pro, Phe, or Val; this leads to other variants. And when serine at the fifth position of PRS is replaced by, for example, Tyr, Trp, Thr, optionally also by Cys, or Met, a further type of variant is present. According to the invention, those sequences that contain at least at one of the positions 1, 2, 4, and 5 of the PRS sequence an amino acid residue that is different from Val-1, Pro-2, Gly-4, and/or Ser-5 are referred to as variants of the pentapeptide sequence.

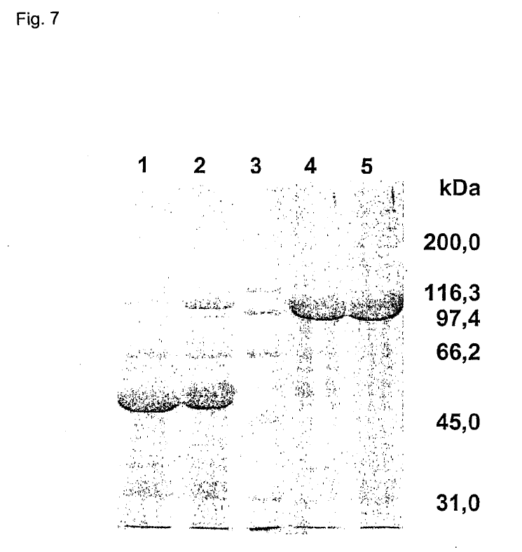

When the LH_{N} fragment of BoNT(A) (or the complete toxin) or any other protein/polypeptide, normally obtained by recombinant expression as a single-chain protein/polypeptide but is biologically/biochemically active (only) in the dichain form, contains the pentapeptide sequence Val-Pro-Xaa-Gly-Ser (wherein Xaa is any of the 20 naturally occurring amino acids and wherein the four other amino acids can be replaced in accordance with the meaning of the preceding paragraph), it will be present in the lysate of the E. coli host cells (for example, E. coli K12, in particular, E. coli K12 host cells of the strains M15[pREP4], XL1-BLUE or UT5600) in the dichain form, wherein in the case of BoNT(A) the light chain is covalently bonded to H_{N} or the complete heavy chain by a disulfide bridge (FIG. 7). The cleavage of the polypeptide chain is realized either directly after cell lysis or is completed substantially after several hours of incubation of the cell lysate. An auto-proteolysis by the activity of the protease domains of the toxin or toxin fragment can be excluded because the protease-inactive mutants that are modified accordingly in the loop area are also present in the dichain structure after expression and disintegration of the E. coli host cells. Obviously, a protease of the E. coli host strain is responsible for the cleavage of the PRS pentapeptide sequence.

A further preferred modification according to the paragraph beginning “Surprisingly, the inventor has . . . ” four paragraphs earlier (on page 6) resides in that N-terminal of the PRS sequence at a spacing of 1 to 20 amino acid residues (the amino acid in the direction of the N-terminal end that is located immediately adjacent the valine residue of the pentapeptide PRS sequence, in the case of the FIG. 3-2 to FIG. 3-5 a leucine residue, has a spacing of 1 amino acid residue from the PRS sequence), in particular, at a spacing of 3 to 15 amino acid residues, especially at a spacing of 3 to 10 amino acid residues, particularly preferred at a spacing of 3 to 8 amino acid residues, and even more preferred at a spacing of 3 amino acid residues, a basic amino acid residue, preferably a lysine residue or arginine residue, is present wherein at its C-terminal end the protease of the E. coli host cell cleaves the loop sequence. After cleavage, a polypeptide is thus obtained that, for example, has two amino acid residues (when the above defined spacing is 3 amino acid residues)—terminal from the valine residue of the PRS sequence. In the present case, “modification” does not necessarily mean a modification in the true sense, i.e., an insertion or substitution of an amino acid residue, so that subsequently N-terminal of the PRS sequence in the afore defined spacing of 1 to 20 amino acid residues a basic amino acid residue (for example, a lysine residue) is located. It is only important that a basic amino acid residue (such as a lysine residue or arginine residue) is present N-terminal of the PRS sequence at the aforementioned spacing.

Another modification, also not mandatory but preferred, in accordance with the paragraph “Surprisingly, the inventor has . . . ” five paragraphs earlier resides in that the loop sequence in which the protease of the E. coli host cells cleaves has a length of at least nine amino acid residues. Preferred lengths of the loop sequences are at least 12, at least 15, at least 18, at least 20, and at least 23 amino acid residues. Particularly preferred lengths of the loop sequence are 15 to 22, in particular, 18 to 22 amino acid residues.

The method according to the invention is in very general terms a method for producing proteins/polypeptides in dichain form wherein the two chains are disulfide-bridged, by means of recombinant expression in E. coli host cells, wherein (i) the protein/polypeptide exerts its biologic activity as a dichain disulfide-bridged protein/polypeptide; (ii) the C-terminal amino acid residue of the first chain is an Arg residue or Lys residue; (iii) the second chain of the protein/polypeptide has N-terminal of a cysteine residue as the N-terminal end 1 to 20 amino acid residues and a pentapeptide sequence VPXGS designated as PRS, wherein X is any naturally occurring amino acid, wherein V is Val, Leu, Ile, Ala, Phe, Pro or Gly, wherein P is Pro, Leu, Ile, Ala, Phe, Val, or Gly, wherein G is Gly, Leu, Ile, Ala, Pro, Phe, or Val, and wherein S is Ser, Tyr, Trp, or Thr; and (iv) the method comprises the following steps: (a) modification of the protein/polypeptide, at the nucleic acid level, so that the protein/polypeptide in its modified form has within its loop area the aforementioned pentapeptide sequence (VPXGS); (b) insertion of the construct modified at the nucleic acid level into the E. coli cells; (c) cultivation and subsequent lysis of the host cells; and (d) isolation of the dichain proteins/polypeptides.

According to the invention, the first chain of the protein/polypeptide is preferably the chain that is coded by the N-terminal end of the corresponding DNA while the second chain of the protein/polypeptide accordingly is the chain that is coded by the C-terminal end of the corresponding DNA. Since the expression of 5′-DNA-3′ leads to N-polypeptide-C, in the aforementioned preferred case of the invention this means that the expression can be represented as follows: 5′ DNA-3′ expresses to N-first polypeptide chain-C-bop-N-second polypeptide chain-C. According to the invention, the loop is already cleaved in situ so that finally the polypeptide/protein N-first polypeptide chain-C-N-second polypeptide chain-C according to the invention is obtained in dichain structure.

The phrase “the second chain of protein/polypeptide has N-terminal of a cysteine residue as the N-terminal end 1 to 20 amino acid residues and a pentapeptide sequence VPXGS designated as PRS” means that the N-terminal end is not formed, for example, by the valine residue of the pentapeptide sequence VPXGS but by another (any) amino acid residue. Between the latter and the valine residue of the PRS, further 1 to 19 amino acid residues can be located but the N-terminal amino acid residue can be bonded directly, for example, to the valine residue, by means of a peptide bond, i.e., can be an immediate neighbor of the valine residue of the PRS.

The proteins/polypeptides according to the invention that can be isolated in their (biologically) active dichain structure, are proteins whose C-terminal end of the first chain has a basic amino acid residue, in particular, an Arg residue or Lys residue, and whose second chain is provided N-terminal with 1 to 20 amino acid residues and with the pentapeptide sequence VPXGS referred to as PRS wherein X, V, P, G, and S are defined as above.

According to the present invention, in the case of immunotoxins that are based on recombinant ricin, for example, a treatment by a sequence-specific protease such as thrombin or factor Xa for activation is obsolete. For example, in the case of immunotoxins based on diphtheria toxin or pseudomonas toxin a significant increase in efficiency was to be expected, and is actually also obtained, because processing by a protease of the target cell as the rate-determining step for the translocation of the enzymatic domain of the toxins into the cytoplasm is no longer required. Such immunotoxins that are already present as a dichain disulfide-bridged polypeptide can be applied in small doses and still provide the same cell-toxic action. This lowers, on the one hand, the therapy costs and, on the other hand, reduces the risk of the formation of antibodies that would make the immunotoxins ineffective upon further applications. A method for producing dichain disulfide-bridged and thus activated immunotoxins is provided by the present invention. With the method provided according to the invention, it is also possible to prepare fusion proteins or hybrid proteins, i.e., proteins with a peptide tag for the affinity purification, in a dichain form, whose two polypeptide chains are covalently bonded by a disulfide bridge and, after affinity chromatographic or other purification methods, can be separated by simple reduction with thiol-containing substances such as β-mercaptoethanol, DTT, or reduced glutathione.

The recombinant expression of clostridial neurotoxins and its fragments (for example, LH_{N} fragment or a derivative of a clostridial neurotoxin, for example, with modified cell specificity) in expression strains of E. coli such as M15[pREP4] or BL21(DE3) produces single-chain polypeptides. By treatment of these polypeptides with trypsin, cleavage takes place in the area of the loop sequence in the transition area of the protease domain to the translocation domain. Since trypsin is not a sequence-specific protease, cleavage, usually unwanted, in further areas of the polypeptide is probable. For example, BoNT(A) is cleaved by trypsin additionally between H_{N} and H_{C} so that a dichain LH_{N} fragment and H_{C} fragment are produced. In order to ensure selective cleavage in the loop area desired in most cases, the presence, optionally after insertion, of a recognition sequence for specific endoproteases is required.

The cleavage of recombinant fusion proteins/hybrid proteins by means of sequence-specific endoproteases such as thrombin, factor Xa, genenase etc. is within the realm of the generally known spectrum of methods. It is possible to separate, after purification, a fusion partner that imparts improved solubility to a recombinant protein/polypeptide and/or improved expression or serves as a peptide tag for the affinity purification. For this purpose, the protein solution is incubated with a suitable endoprotease in soluble form or in immobilized form on a matrix.

This technique can be utilized also for the expression of the aforementioned recombinant proteins/polypeptides that exert their normal biologic/biochemical activity as a dichain protein/polypeptide but by means of recombinant DNA technology are obtained as inactive single-chain proteins/polypeptide (for example, the expression of clostridial neurotoxins, fragments of clostridial neurotoxins such as LH_{N} fragments or of derivatives of clostridial neurotoxins, for example, with modified cell specificity): A recognition sequence for an endoprotease is cloned into the polypeptide, preferably at the level of the nucleic acids, for example, into the loop area between L and H_{N}, and, moreover, at the N-terminal or C-terminal end a further recognition sequence for the same or a further endoprotease, flanked by a peptide tag for the affinity purification is cloned. The single-chain expressed protein/polypeptide is then activated by treatment with the corresponding endoprotease or endoproteases at the same time or sequentially by cleavage in the loop area between L and H_{N} and the peptide tag is removed.

Aside from the costs for the use of such endoproteases and the thus required additional working steps, their use in pharmaceutical preparations (for example, the use of recombinant botulinum toxins or their derivatives) is highly problematic with regard to pharmaceutical law-based (regulatory) reasons. On the one hand, the purity of the employed endoprotease must be experimentally proved and, on the other hand, a complete removal and particularly virus-freeness of the preparation in the further course of the purification protocol must be documented precisely; this, in general, requires an enormous analytical expenditure. Since in the future also botulinum toxins, for example, with improved properties or modified cell specificity are to be produced by recombinant expression, there is a great need for an expression method that enables providing of the aforementioned recombinant proteins/polypeptides that exert their normal biologic/biochemical activity as dichain proteins/polypeptides but are obtained by means of recombinant DNA technology in the form of inactive single-chain proteins/polypeptides, in particular, enables providing botulinum toxins or their derivatives as dichain disulfide-bridged and thus biologically active polypeptides/proteins without having to use endoproteases.

The invention that will be explained in the following in more detail therefore provides in the broadest sense a method with which proteins such as clostridial neurotoxins as well as their fragments and derivatives can be produced by recombinant expression in E. coli host cells and can be isolated in their dichain disulfide-bridged and thus biologically active form without their activation requiring the addition of an endoprotease.

In a first preferred embodiment of the invention, the amino acid sequence of the loop area of the BoNT(A) between the cystine residues 430 and 454 (see FIG. 3-1 to 3-5) has been modified in that the expressed toxin or its fragments/derivatives in the lysate of the E. coli host cells are already present as a dichain polypeptide. The two chains are covalently bonded to one another with participation of the cystine residue 430 and 454 by means of a disulfide bridge. In a particularly preferred embodiment of the invention, as explained in FIG. 3, the pentapeptide Asp₄₄₃-Asn₄₄₇ (DKGYN) can be replaced by Val-Pro-Arg-Gly-Ser (VPRGS). In further preferred embodiments of the invention, the pentapeptide Asp₄₄₃-Asn₄₄₇ (DKGYN) can also be replaced by Val-Pro-Tyr-Gly-Ser (VPYGS), Val-Pro-His-Gly-Sr (VPHGS) or Val-Pro-Gln-Gly-Ser (VPQGS). In this context, it also holds true that not only the central amino acid residue can be any naturally occurring amino acid but also that the four other amino acid residues can also be exchanged, as has been explained supra in detail (when exchanging at least one of these residues a variant of the PRS sequence is present in the meaning of the invention). Moreover, in regard to this embodiment as well as all other preferred embodiments that will be explained in the following it holds true that additionally it is preferred when the loop sequence has, N-terminal to PRS at a spacing of 1 to 28 amino acids, a basic amino acid residue, especially a lysine or arginine residue.

It is easily apparent to a person skilled in the art that further exchanges of individual or several amino acid residues or the insertion or deletion of further amino acid residues in the area of the above characterized loops of BoNT(A) also leads to the result that the expressed toxin according to the invention or the fragments/derivatives derived therefrom in the lysate are present as dichain polypeptides. These possible variants are also encompassed by the present invention.

It is also easily apparent to a person skilled in the art that the pentapeptide Asp₄₄₃-Asn₄₄₇ (DKGYN) present in the wild type of BoNT(A) can be replaced by a hexapeptide, by a heptapeptide, by an octapeptide etc. as long as in the expressed and single-chain translated polypeptide/protein the PRS-pentapeptide sequence or one of its conceivable variants is present within the loop area. As has been explained above, it is preferred when N-terminal of the pentapeptide a basic amino acid residue (preferably lysine) is present.

It is furthermore apparent to a person skilled in the art that the preferred embodiment of the pentapeptide (Val-Pro-Arg-Gly-Ser) of the PRS is a part of a possible recognition sequence for the protease thrombin that plays an important role in the cascade of blood coagulation and has a high sequence specificity. It is expressly pointed out that, firstly, neither in the botulinum neurotoxin type A nor in other polypeptides a cleavage by thrombin is required in order to obtain the desired dichain disulfide-bridged form and that secondly, the thrombin recognition sequence in itself, i.e. in its unmodified form, is beneficial for cleavage by the protease activity of the E. coli lysate but is not at all required. Embodiments of the PRS pentapeptide sequences that are inserted or generated in the corresponding polypeptides (better: in their loops) that do not contain the arginine residue at whose C-terminal end thrombin can cleave (instead, another naturally occurring amino acid is present) also lead to cleavage in the loop, as has been explained above. The cleavage is realized preferably at a lysine residue of the loop that is N-terminal to the pentapeptide, as has been explained above (see also example 2; FIG. 3).

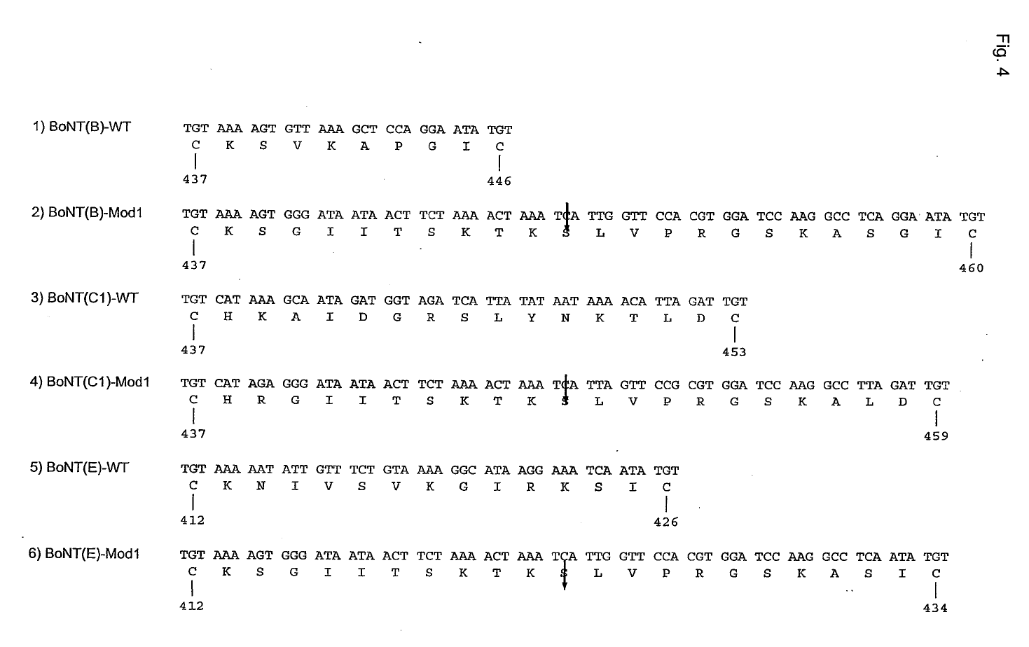

Since other serotypes of the botulinum toxin, such as BoNT(B) and BoNT(C1) as long-acting and the BoNT(E) as short-acting neurotoxins, as well as entirely different polypeptides/proteins that can be recombinantly expressed as a single chain but exert their biologic activity only as a dichain can be utilized therapeutically, it would be desirable that these neurotoxins as well as fragments or derivatives thereof (and also the other polypeptides/proteins) could also be obtained as dichain disulfide-bridged polypeptide/proteins from E. coli lysates In particular in the case of BoNT(B), a complete cleavage of the recombinant toxin in E. coli lysate to the dichain polypeptide/protein would provide a significant advantage in comparison to the native neurotoxins that is secreted in Clostridium botulinum that, in general, is at least 40 percent present as a single-chain and thus inactive polypeptide and cannot be separated from the active dichain form. It is also apparent that the loop areas of the neurotoxins of the serotypes B, C1, and E between the cysteine residues participating in the disulfide bridge relative to the loop of BoNT(A) are significantly shorter (FIGS. 3 and 4). While in the case of BoNT(A) 23 amino acid residues (Val₄₃₁-Leu₄₅₃) are present, in BoNT (B) only 8 (Lys₄₃₈-Ile₄₄₅) in BoNT(C1) 15 (His₄₃₈-Asp₄₅₂), and in BoNT(E) 13 amino acid residues (Lys₄₁₃-Ile₄₂₅) are present in this region. In spite of this, with the exception of BoNT(B), it was found that these comparatively shortened regions are sufficiently long in order to enable cleavage of the chain and the formation of disulfide bridges when they have the PRS sequence in accordance with the present invention. Even though the BoNT(B), when a pentapeptide in the loop is exchanged for a PRS pentapeptide sequence (thus, entire length of the loop sequence only eight amino acid residues), was cleaved into two chains (light and heavy) in the meaning of the invention, better results were obtained, i.e., it is preferred in accordance with the invention, to have a loop of at least 9, at least 15, at least 20, or even at least 22 amino acid residues. One of the last-mentioned embodiments in which the loop has 22 amino acid residues, is explained in an exemplary fashion by the sequences of FIGS. 4-1 and 4-2 or a comparison between these two.

It has also been experimentally proved that an exchange of the loop areas in the subtypes B, C1, etc. or significant parts thereof for the loop area of BoNT(A), or significant parts thereof, would be preferred with regard to the cleavage of the neurotoxins to disulfide-bridged dichain polypeptides/proteins, in particular when in this way the loop is extended to at least 9, preferred 15, residues and/or N-terminal of PRS a basic amino acid residue (for example, and preferred, a Lys residue) has been inserted (inasmuch as beforehand no N-terminal basic or Lys residue was present). Especially preferred are changes as illustrated in FIG. 4 (wherein the PRS sequences in FIG. 4 are VPRGS, but at the same time, and preferred as well, are however the sequences VPYGS, VPHGS, VPQGS, VPKGS, VPIGS and VPAGS).

In other embodiments of the invention the amino acid sequences and the gene portions coding therefore of the loop areas in the botulinum toxins of the serotypes B, C1, D, E, F, and G as well as of the tetanus toxin are modified between the cysteine residues participating in the disulfide bridge between L and H_{N} in that the expressed toxins or the fragments/derivatives derived therefrom in the lysate of E. coli host cells are already present as dichain polypeptides in which the two chains are covalently bonded by a disulfide bridge (the same holds true also for any other polypeptides/proteins that are generated by recombinant expression as a single chain but develop biologic activity only in the dichain form). In preferred embodiments of the invention, the complete loop areas (or parts thereof) of the neurotoxins or of the toxin fragments/derivatives derived therefrom can be exchanged for the complete loop area of BoNT(A), as characterized in FIG. 3, or parts of the loop area of BoNT(A), wherein the pentapeptide Asp₄₄₃-Asn₄₄₇ is replaced preferably e.g. by Val-Pro-Arg-Gly-Ser (VPRGS). In further preferred embodiments of the invention, the pentapeptide Asp₄₄₃-Asn₄₄₇ can also be replaced by Val-Pro Tyr-Gly-Ser, Val-Pro-His-Gly-Ser, or Val-Pro-Gln-Gly-Ser. In especially preferred embodiments of the invention, the loop areas or parts of the loop areas of the aforementioned neurotoxins and the fragments/derivatives derived therefrom can be replaced by the oligopeptide Arg/Ser-Gly-Ile-Ile-Thr-Ser-Lys-Thr-Lys-Ser-Leu-Val-Pro-Arg-Gly-Ser-Lys-Ala (18mer: R/SGIITSKTKSLVPRGSKA). Further exchanges, insertions or deletions of individual or several amino acid residues in the area of the above described loop sequence, as shown e.g. in FIG. 4, that also lead to the expressed neurotoxin or its fragment/derivative after the expression in E. coli (for example, in E. coli K12 host cells or its derivatives) as disulfide-bridged dichain polypeptide/protein are expressly encompassed by this invention (the same holds true also for any other polypeptides/proteins that can be generated by recombinant expression as a single chain but have biologic activity only in the dichain form).

As has been repeated frequently above, with the method according to the invention according to a further embodiment of the invention, fusion proteins or hybrid proteins can be produced also which have, for example, the following components A, B, and C:
an effector domain that, by its enzymatic activity, is able e.g. to inhibit secretion in target cells or kill them (A);
a loop sequence that, according to the invention as explained above, is modified and that has the above defined PRS pentapeptide sequence VPXGS (for example, a modified loop sequence of BoNT(A) or variants thereof as illustrated in FIG. 3) and that may have attached a cysteine residue N-terminally and/or C-terminally (B); as well as
a cell binding domain that imparts a cell specificity to the fusion protein or hybrid protein (C).

The component B (loop sequence) can also be in both immediately aforementioned embodiments preferably likewise (i) a modified loop sequence as illustrated in FIG. 4, (ii) any of the sequences derived therefrom inasmuch as the central residue of PRS may be the residue of any naturally occurring amino acid, or (iii) a variant (see above for definition of variant) of (i) or (ii). In FIG. 4, the respective loop sequences of BoNT(B), BoNT(C1) or BoNT(E) with the exception of one or two N-terminal and the two C-terminal amino acid residues have been deleted and the deleted amino acid residues have been replaced by the 17mer GIITSKTKSLVPRGSKA (FIGS. 4-2 and 4-6) or the 18mer RGIITSKTKSLVPRGSKA (FIG. 4-4) of the modified loop sequence of BoNT(A).

In addition to the aforementioned components A, B and C, the fusion/hybrid proteins can have a translation domain (which in the case of the botulinum neurotoxins is located between the loop sequence and the cell binding domain). This additional domain assists in the insertion of the effector domain into the cytoplasm of the target cell. The expression of such fusion proteins in E. coli (for example, E. coli K12 or derivatives thereof) leads to dichain polypeptide/proteins in which one domain is on one chain and the two other domains are on the second chain (in the case of the botulinum toxins the effector domain on the light chain is covalently bonded by a disulfide bridge to the two other domains on the heavy chain.

These inventive fusion or hybrid proteins can be so-called immunotoxins that in particular find use in tumor therapy. In this connection, the toxin domain is imparted a specificity for a certain cell type, in general, a tumor cell, by attaching a cell binding domain. As a toxin domain, primarily the enzymatic domains of diphtheria toxin, pseudomonas toxin, and ricin are used. These toxins belong to the dichain AB toxins in which the A-chain that provides the enzymatic activity is bonded by a disulfide bridge covalently to the B-chain that combines the translocation activity and cell binding activity. However, other toxins or toxin fragments in immunotoxins are conceivable inasmuch as the desired action (for example, killing off tumor cells) is developed in the target cells. While the first generation of immunotoxins have been produced by chemical coupling of the toxin domain as, for example, the A-chain of ricin, with a monoclonal antibody, the immunotoxins of the second generation are produced by recombinant expression as Fab toxins, single-chain Fv toxins (scFv toxins) or disulfide-stabilized Fv (dsFv toxins) but also as fusion proteins with growth factors or cytokines primarily in E. coli (Reiter, 2001). In future generations of immunotoxins the cell specificity can also be imparted by modified polypeptides that are selected in accordance with high affinity binding to, for example, tumor-specific surface protein, for example, of the protein families of affilins, ankyrin repeat proteins, or anticalins.

In all conceivable variants of the immunotoxins it must be ensured that the enzymatic toxin domain can pass into the cytoplasm of the target cell in order to develop therein the toxic action. Since the immunotoxins in E. coli is expressed as a single-chain polypeptide, a proteolytic cleavage as well as a reduction of a disulfide bridge are required in order to separate, with regard to the chains, the enzymatic toxin domain from the translocation unit and the cell binding domain. In the case of recombinant diphtheria toxin fragments and the recombinant pseudomonas exotoxin fragment, cleavage occurs after internalization in the endosomal compartment of the target cell by a cellular protease such as furin (Williams et al., 1990). Ricin, on the other hand, has no such processing site and requires therefore an artificially inserted protease recognition sequence in order for it to be administered as an already dichain disulfide-bridged immunotoxin. However, in the case of immunotoxins that are based on diphtheria toxin and pseudomonas exotoxin, only a minimal portion of the internalized fusion proteins is cleaved so that only an equally minimal portion of the enzymatic domains can reach the cytoplasm (Ogata et al., 1990). The subsequently presented preferred embodiments of the invention describe methods and constructs wherein by means of the methods variants of immunotoxins, as described in the preceding paragraphs, are produced by recombinant expression in E. coli host cells and can be isolated in their dichain disulfide-bridged and thus biologically (enzymatically) active form without their activation requiring a cellular endoprotease or an endoprotease added in vitro. These immunotoxins are capable of transporting the enzymatic toxin domain into the target cell in a translocation competent form so that cleavage by a cellular protease is not required and significantly reduced doses of immunotoxins may be employed in order to achieve the desired cell toxic effects.

A further preferred embodiment of the invention comprises accordingly further a fusion protein or hybrid protein that has the following components A, B, and C:
a toxin domain or its fragment/derivative (A);
a loop sequence that according to the invention as described above is modified and has the above defined PRS pentapeptide sequence VPXGS (for example, one of the modified loop sequences of BoNT(A) illustrated in FIG. 3 or variants thereof) and that may have attached thereto N-terminally and/or C-terminally a cysteine residue (B); as well as
a cell binding domain that can be taken from a representative of the protein families of monoclonal antibodies, their fragments, of affilins, of ankyrin repeat proteins, of anticalins, of growth factors (for example, TGF-alpha, FGF, VEGF, or IGF-1) or of the cytokines (for example, IL2, IL4, or IL6) (C).

In accordance with this last preferred embodiment, the component B (loop sequence) can be likewise (i) one of the modified loop sequences illustrated in FIG. 4, (ii) any sequence derived therefrom inasmuch as the central residue of PRS may be the residue of any naturally occurring amino acid, or (iii) a variant (see above for definition of variant) of (i) or (ii).

The toxin domain can be the A-chain of ricin, a fragment of the pseudomonas exotoxin such as PE40 or PE38 (domains II and III with or without domain Ib; FIG. 2) or a fragment of the diphtheria toxin. The aforementioned effector or toxin and cell binding domains are to be understood as examples only. All proteins or protein fragments are encompassed by the invention that, on the one hand, impart to the fusion protein/hybrid protein a specific binding activity to a surface antigen of a target cell, for example, a tumor cell, and, on the other hand, in a target cell after internalization exert a certain action, for example, killing off the cell, wherein the expression of such fusion/hybrid proteins according to the invention in E. coli produces dichain polypeptides/proteins in which the toxin domain or derivatives thereof are covalently bonded by a disulfide bridge to the cell binding domain.

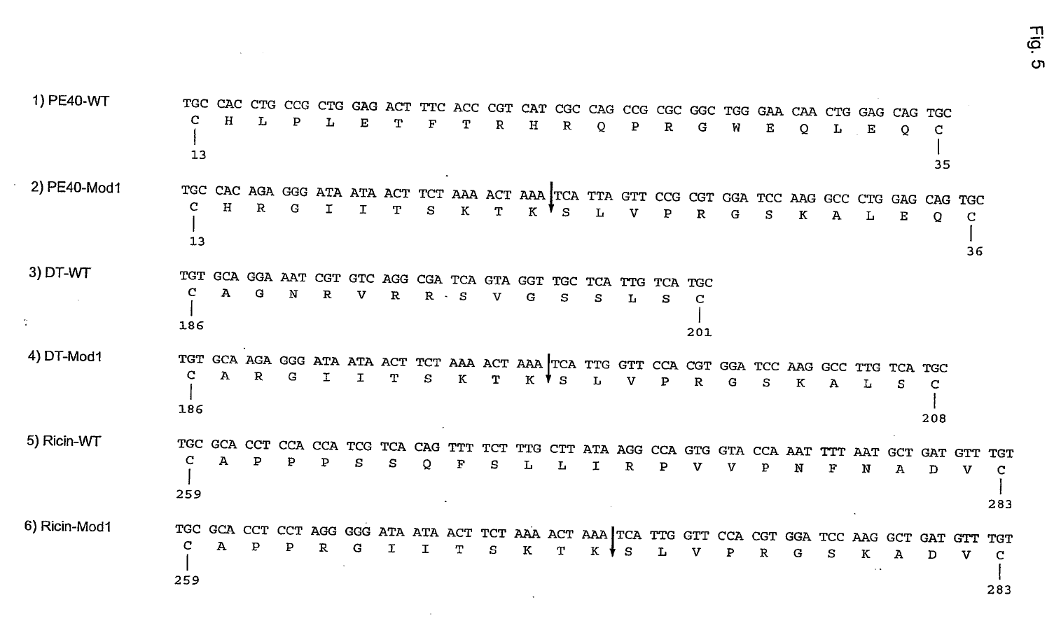

For improving the efficiency and specificity of immunotoxins based on pseudomonas exotoxin different approaches have been selected in the past. For example, the receptor binding domain (domain Ia with the amino acid residues 1-152) has been exchanged for fragments of a monoclonal antibody and at the same time the loop area (FIGS. 2 and 5) in the translocation domain (domain II) between the cysteine residues 13 and 35 (numbering relative to domain II) has been modified such that the latter no longer was sensitive to cleavage of the ubiquitous cellular protease furin but instead to special proteases that are expressed to a greater degree and partially secreted only by certain tumor cells (U.S. Pat. No. 6,426,075). This modified protease sensitivity was designed to impart to the immunotoxins an increased cell specificity in addition to the exchanged receptor binding domain. However, it is not to be expected that an increased cleavage in the loop and thus improved translocation efficiency of the enzymatic domain III will result by means of other cellular proteases.

According to a further approach for an immunotoxin, the receptor binding domain and the N-terminal area of the translocation domain were removed up to the arginine residue 27 within the loop area. The required cell specificity in such an immunotoxin was imparted, for example, by insertion of a V_{H} domain of a monoclonal antibody to which was bonded the V_{L} domain by means of a disulfide bridge at the site of the Ib domain between the domains II and III or by attachment of the C-terminal end of the domain III (U.S. Pat. No. 5,980,895). In such constructs an activation via protease is no longer required; on the one hand, this should effect a significantly increased transportation efficiency. However, on the other hand, it is to be expected that the translocation by means of the receptor binding domains located N-terminally or C-terminally of the enzymatic domain III will be impaired like the V_{H} domain of a monoclonal antibody or TGF-alpha. Because these receptor binding domains are not separated from the enzymatic domain, negative effects on the enzymatic activity and thus toxicity in the target cells are to be expected. A relative maximal degree of cytotoxic activity is obtained with a pseudomonas exotoxin-based immunotoxin when, on the one hand, the loop between the cysteine residues 13 and 35 is already present in the cleaved disulfide-bridged form and an activation by a cellular protease is therefore not required, and when, on the other hand, the receptor binding domain is fused in place of the domain I of the exotoxin to the N-terminal end of the translocation domain so that, after reduction in the cytoplasm, it is separated from the toxin domains and therefore cannot impair the enzymatic activity of the domain III.

An especially preferred embodiment of the invention comprises therefore a fusion/hybrid protein comprising a cell binding domain that can be taken from a representative of the protein families of monoclonal antibodies, their fragments, of affilins, of ankyrin repeat proteins, of anticalins, of growth factors (for example, TGF-alpha, FGF, VEGF, or IGF-1) or the cytokines (for example, IL2, IL4, and IL6), to which is fused C-terminally a modified PE38 fragment that can carry at the extreme C-terminal end the retention signal for the endoplasmatic reticulum, Lys-Asp-Gly-Leu, or variants thereof. The modification of the PE38 fragment consists of the complete loop sequence (or only a part thereof) between the cystine residues 13 and 35 having been exchanged for the PRS pentapeptide sequence VPXGS, preferably for the modified loop sequence of BoNT(A) illustrated in FIG. 3 or variants thereof, in particular for the peptide sequence Arg-Gly-Ile-Ile-Thr-Ser-Lys-Thr-Lys-Ser-Leu-Val-Pro-Arg-Gly-Ser-Lys-Ala (FIG. 5) (see above for definition of variants). Preferably, in this embodiment it is also ensured that a basic amino residue is located N-terminally to PRS at a spacing of 1 to 20 amino acid residues, as illustrated in the sequence of FIG. 5. A correspondingly modified PE38 fragment as well as fusion/hybrid proteins that contain this modified fragment are present in the lysate of the E. coli host cells (for example, M15[pREP4]) in the dichain disulfide-bridged form.

In contrast to the pseudomonas exotoxin, the enzymatic domain of the diphtheria toxin, the A-chain, is present at the N-terminal end. On the C-terminal B-chain the translocation domain and the receptor binding domain are present. Both chains are connected by a loop sequence in which at the arginine residue 193 upon secretion from cells of Corynebacterium diphtheriae a proteolytic cleavage takes place by a protease (Collier, 2001). The two chains after cleavage remain covalently bonded to one another by a disulfide bridge between the cysteine residues 186 and 201. In this regard, the diphtheria toxin is similar in its domain structure to the botulinum toxins and the tetanus toxin.

For producing recombination immunotoxins, the receptor binding domain or a part thereof was exchanged, for example, for VEGF or IL2 (Arora et al., 1999; Williams et al., 1990) in order to impart to the fusion protein a new cell specificity. In order for the A-chain to reach the cytoplasm of the target cells, on the one hand, the polypeptide chain of the immunotoxin expressed as a single chain in E. coli must be cleaved in the area of the loop between the A-chain and the B-chain and, on the other hand, the disulfide bridge must be reduced. While the latter occurs in the course of the translocation process, the proteolytic cleavage by a cellular protease is incomplete so that only a minimal portion of the A-chains can be released into the cytoplasm (Williams et al., 1990). If the immunotoxin were present in the dichain disulfide-bridged form already at the time of administration, a significant efficiency increase could be expected because all A-chains would be made available in a translocation-competent form.

A further especially preferred embodiment of the invention comprises therefore a fusion or hybrid protein comprising a cell binding domain that can be taken from a representative of the protein families of monoclonal antibodies, their fragments, of affilins, of ankyrin repeat proteins, of anticalins, of growth factors (for example, TGF-alpha, FGF, VEG, or IGF-1) or of the cytokines (for example, IL2, IL4, or IL6) to which is fused at the N-terminal end a modified diphtheria toxin fragment. This toxin fragment can comprise the A-chain as well as at least one translocation domain of the B-chain (Gly₁-Phe₃₈₉ or Gly₁-Asn₄₈₆). The modification of the diphtheria toxin fragment consists in that the complete loop sequence (or only a part thereof) between the cysteine residues 186 and 201 is exchanged for the modified loop sequence of BoNT(A) illustrated in FIG. 3 or variants thereof, in particular for the peptide sequence Arg-Gly-Ile-Ile-Thr-Ser-Lys-Thr-Lys-Ser-Leu-Val-Pro-Arg-Gly-Ser-Lys-Ala (FIG. 5) (see above for definition of the variants). A correspondingly modified diphtheria toxin fragment as well as fusion proteins that contain this modified fragment are present in the lysate of the E. coli host cells as, for example, M15[pREP4] in the dichain disulfide-bridged form.

Ricin-based immunotoxins of the first generation were produced by linking the A-chain of the ricin with a monoclonal antibody. This was achieved in the past by derivatization of the antibody with a chemical linker molecule that formed a disulfide bridge with the thiol function of the cysteine residue located at the C-terminal end of the A-chain. Such conjugates were heterogenous because of the undirected derivatization of the antibody. The efficiency against tumors was insufficient, not the least because of the size of the conjugate and the lack of the translocation domain localized at the B-chain. When the B-chain in the native form is also present as a component of the immunotoxin, the toxicity is significantly increased but, as a result of the lectin-like cell binding properties of the B-chain, unspecific uptake into other than the desired target cells takes place also. This target conflict was countered by a strategy according to which the B-chain was modified such that the translocation activity remained intact but the binding affinity for glyco structures at the cell surfaces was however significantly reduced (patent application WO 89/04839). Recombinant expressed immunotoxins that contain such a modified B-chain are however of a single-chain structure so that, as a result of the lack of recognition sequence for a cellular protease in the linker peptide between A-chain and B-chain, release and translocation of the A-chain upon uptake of the immunotoxins into the target cell are not possible at all or possible only very inefficiently. In U.S. Pat. No. 6,593,132 modifications of this native linker peptide are documented that represent recognition sequences for different cell-specific proteases. Ricin variants with such modifications should have a corresponding cell specificity inasmuch as the respective protease that can proteolytically cleave the modified linker peptide is expressed only in the desired target cells in comparison to other cell types in significantly increased quantities. However, it must be assumed that the cleavage is taking place only in a fraction of the internalized toxin molecules and thus also only a corresponding minimal quantity of A-chains is translocated into the cytoplasm. Desirable would be ricin-based dichain immunotoxins in which the A-chain is linked by a disulfide bridge to a modified B-chain in which the translocation activity remains intact but the unspecific pectin-like cell binding properties are suppressed and that are fused at their C-terminal end with a specific cell binding domain. Such immunotoxins would combine cell specificity and high toxicity.

A further preferred embodiment of the invention comprises therefore a fusion protein that has the following components A, B, and C:
the A-chain of ricin (A);
a loop sequence that is modified according to the invention as described above and has the above defined PRS pentapeptide sequence VPXGS (for example, one of the loop sequences of BoNT(a) illustrated in FIG. 3 or variants thereof) and that may have attached N-terminally and/or C-terminally a cysteine residue (B), as well as
a cell binding domain that can be taken from a representative of the protein families of the monoclonal antibodies, their fragments, of affilins, of ankyrin repeat proteins, of anticalins, of growth factors (for example, TGF-alpha, FGF, VEGF or IGF-1) or of cytokines (for example, IL2, IL4, or IL6) (C).

The component B according to this last preferred embodiment can be likewise (i) one of the modified loop sequences illustrated in FIG. 4, (ii) any sequence derived therefrom as the central residue of PRS can be the residue of any naturally occurring amino acid, or (iii) the variant (see above for definition of variant) of (i) or (ii)).

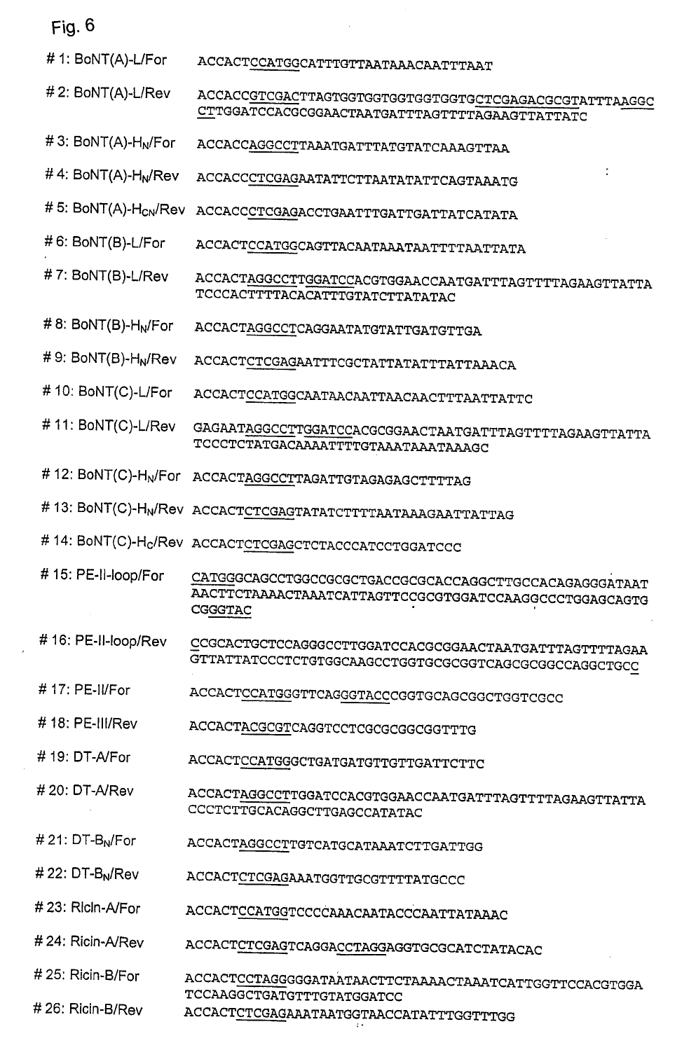

In particular, the loop sequence can contain the peptide sequence Ala-Pro-Pro-Arg-Gly-Ile-Ile-Thr-Ser-Lys-Thr-Lys-Ser-Leu-Val-Pro-Arg-Gly-Ser-Lys-Ala-Asp-Val (FIG. 5-6), i.e., a modified loop of the A-chain of ricin. A cysteine residue is preferably additionally provided C-terminally at the loop sequence. In the PRS sequence Val-Pro-Arg-Gly-Ser contained therein, Arg can however be any other naturally occurring amino acid Xaa. At both ends, the loop sequence can be expanded by further amino acid residues (for example, glycine and serine residues). Moreover, the A-chain of the ricin can be linked with the complete B-chain, or parts or variants thereof, by a loop sequence that replaces the amino acid residues between the cysteine residues 259 and 283 of the wild type sequence of the pro ricin entirely or partially and at least encompasses the area of the modified BoNT(A) loop described in FIG. 3 or variants thereof. In this connection, a disulfide bridge is formed by the cysteine residues 259 and 283 (relative to the wild type sequence of the pro ricin). A cell binding domain is fused to the C-terminal end of the B-chain and is taken from the above mentioned polypeptide families. Corresponding fusion/hybrid proteins are present in the lysate of the E. coli host cells, for example, of cells of the strain M15[pREP4], in the dichain disulfide-bridged form.

A further embodiment of the invention concerns recombinant fusion proteins that have the following components A, B, and C:
a protein or oligo peptide that imparts to the fusion protein a better solubility, effects a higher expression rate and/or enables affinity purification (for example, glutathione-S-transferase (GST), maltose binding protein (MBP), His tag, StrepTag, FLAG tag (A);
a loop sequence that is modified according to the invention as described above and comprises the above defined PRS pentapeptide sequence VPXGS (for example, a modified loop sequence of BoNT(A) illustrated in FIG. 3 or variants thereof) and that may have attached N-terminally and/or C-terminally a cysteine residue, as well as
any type of polypeptide (C).

The component B (loop sequence) in accordance with this last preferred embodiment can be likewise (i) one of the modified loop sequences illustrated in FIG. 4, (ii) any sequence derived therefrom as the central residue of PRS may be the residue of any naturally occurring amino acid, or (iii) a variant (see above for definition of variant) of (i) or (ii)).

In particular the loop can have the peptide sequence Val-Arg-Gly-Ile-Ile-Thr-Ser-Lys-Thr-Lys-Ser-Leu-Val-Pro-Arg-Gly-Ser-Lys-Ala-Leu-Asn-Asp-Leu wherein Arg at the center of PRS can again be Xaa. At both ends it can be expended by further amino acid residues (for example, glycine and serine residues). The expression of such fusion proteins in E. coli leads to dichain polypeptides/proteins whose two chains are covalently bonded by a disulfide bridge and, after completed purification, can be separated from one another without addition of protease after a simple reduction by thiol-containing substances (for example β-mercaptoethanol, DTT, or reduced glutathione). Such an expression system is particularly suitable for recombinant proteins that are to be provided at one of the two terminal ends with a cysteine residue in order to provide, after purification and separation of the fusion partner with the reactive thiol group, a site for e.g. coupling reactions with thiol-reactive linker molecules or modifications with, for example, polyethylene glycol.

The invention comprises moreover all nucleic acids that code for the polypeptides according to the invention described in the preceding sections, taking into consideration the different possibilities of codon use. Moreover, the invention encompasses commercially available or individually constructed cloning and expression plasmids that contain the coding DNA sequences for the respective polypeptides according to the invention as well as suitable cloning and expression strains of E. coli that are transformed with the corresponding expression plasmids and that can express the respective polypeptides according to the invention in their active dichain disulfide-bridged form. One example for such an expression system is an expression plasmid of the pQE series in combination with the E. coli host strain M15[pREP4].

For a person skilled in the art who deals in particular with the development of pharmaceutically useable polypeptides/proteins, the advantages that are related to the fact that for activation of these polypeptides/proteins no endoproteases must be added are clearly apparent. The greatest part of the polypeptides/proteins according to the invention described in preceding sections are particularly targeted for pharmaceutical use. The invention therefore also encompasses pharmaceutical preparations that comprise one of the inventive polypeptides/proteins or a mixture of the inventive polypeptides/proteins as active ingredients as well as useful additives that impart to the preparation a sufficient stability and whose composition is matched to the desired form of administration.

The attached Figures and sequences of the sequence listing are described as follows:

FIG. 1 shows a schematic illustration of the release of botulinum neurotoxin type A with wild type loop or modified loop according to the invention from Clostridium botulinum or Escherichia coli K12. A: in the lysis of Clostridium botulinum cells the neurotoxin is cleaved in the loop area between light chain (L) and heavy chain (H) by a clostridial endoprotease. Both chains are connected to one another by a disulfide bridge. B: After expression of a recombinant neurotoxin with a wild type loop in E. coli and lysis of the cells it is present in the single chain form. C: When a recombinant neurotoxin with loop modified according to the invention is released from E. coli cells, cleavage in the loop area is done by an endoprotease.

FIG. 2 shows a schematic illustration of different recombinant toxins with wild type loop areas as well as loop areas modified according to the invention in comparison after their release from E. coli cells. A: botulinum neurotoxins; B: pseudomonas exotoxin; C: diphtheria toxin.

FIG. 3 shows a comparison of the wild type loop with a selection of loop sequences of BoNT(A) modified according to the invention. Illustrated are nucleotide sequences and the derived amino acid sequences that include the limiting cysteine residues of the light chain and heavy chain. The arrow marks the cleavage site for the endoprotease in E. coli lysate.

FIG. 4 shows a comparison of the wild type loop with an exemplary loop sequence modified according to the invention of the botulinum neurotoxins of the serotypes B, C1, and E, respectively. Illustrated are the nucleotides sequences and the derived amino acid sequences that include the limiting cysteine residues of the light chain and heavy chain. The arrow marks the cleavage site for the endoprotease in the E. coli lysate.

FIG. 5 shows a comparison of the wild type loop with an exemplary loop sequence modified according to the invention of fragment PE40 of the pseudomonas exotoxin, diphtheria toxin (DT), and ricin, respectively. Illustrated are nucleotide sequences and the derived amino acid sequences that includes the limiting cysteine residues. The arrow marks the cleavage location for the endoprotease in the E. coli lysate.

FIG. 6 shows a combination of the oligonucleotides that were used for cloning the recombinant toxins and toxin fragments. Recognition sequences for the restriction endonucleases are underlined.

FIG. 7 shows an analysis of the recombinant LH_{N} fragments of BoNT(A) with bop sequence modified according to the invention on SDS polyacrylamide gel. The expression of the LH_{N} fragment was realized in M15[pREP4] cells that were transformed with the plasmid pQE-BoNT(A)-L_{mod1}H_{N}. Lanes 2 and 5: LH_{N} fragment purified on Ni-NTA agarose; lanes 1 and 4: LH_{N} fragment after incubation with thrombin; trace 3: molecular weight marker. Sample application under reducing conditions (lanes 1 and 2) and non-reducing conditions (lanes 4 and 5).

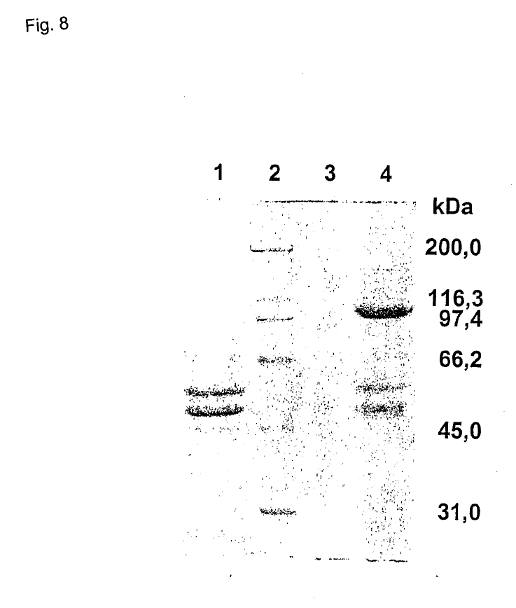

FIG. 8 shows an analysis of the recombinant LH_{N} fragment of BoNT(B) with loop sequence modified according to the invention on SDS polyacrylamide gel. The expression of the LH_{N} fragment is realized in M15[pREP4] cells that were transformed by plasmid pQE-BoNT(B)-L_{mod1}H_{N}. Lanes 1 and 4: fragment LH_{N} purified on Ni-NTA agarose; lane 2: molecular weight marker; lane 3: no application. Sample application under reducing conditions (lane 1) and non-reducing conditions (lane 4).

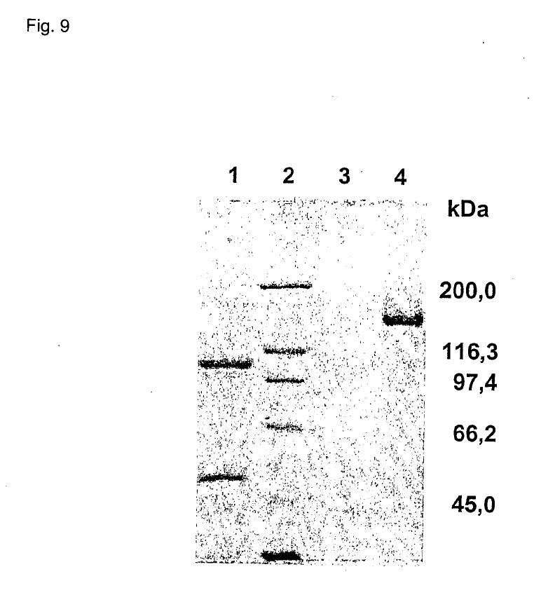

FIG. 9 shows an analysis of recombinant BoNT(C1) with loop sequence modified according to the invention in SDS polyacrylamide gel. The expression of the toxin is done in M15[pREP4] cells that are transformed by the plasmid pQE-BoNT(C1)-L_{mod1}H_{N}H_{C}. Lanes 1 and 4: toxin purified on Ni-NTA agarose; lane 2: molecular weight marker; lane 3: no application. Sample application under reducing conditions (lane 1) or non-reducing conditions (lane 4).

SEQ ID NO. 1 is an example of a nucleic acid (DNA) that codes for a recombinant botulinum neurotoxin type A with loop sequence modified according to the invention and C-terminal hexahistidine tag (rBoTN(A)-mod1).

SEQ ID NO. 2 is an example of a recombinant botulinum neurotoxin type A with loop sequence modified according to the invention and C-terminal hexahistidine tag (rBoTN(A)-mod1).

SEQ ID NO. 3 is an example of a nucleic acid (DNA) that codes for a recombinant LH_{N} fragment of the botulinum neurotoxin type A with loop sequence modified according to the invention and C-terminal hexahistidine tag (rBoTN(A)-L_{mod1}H_{N}). The sequence corresponds to SEQ ID NO. 1 wherein the nucleotides 2620-3888 are deleted.

SEQ ID NO. 4 is an example for a recombinant LH_{N} fragment of the botulinum neurotoxin type A with loop sequence modified according to the invention and C-terminal hexahistidine tag (rBoTN(A)-L_{mod1}H_{N}). The sequence corresponds to SEQ ID NO. 2 wherein the amino acid residues 874-1296 are deleted.

SEQ ID NO. 5 is an example of a nucleic acid (DNA) that codes for a recombinant LH_{N}H_{CN} fragment of the botulinum neurotoxin type A with loop sequence modified according to the invention and C-terminal hexahistidine tag (rBoTN(A)-L_{mod1}H_{N}H_{CN}). The sequence corresponds to SEQ ID NO. 1 wherein the nucleotides 3286-3888 are deleted.

SEQ ID NO. 6 is an example of a recombinant LH_{N}H_{CN} fragment of the botulinum neurotoxin type A with loop sequence modified according to the invention and C-terminal hexahistidine tag (rBoTN(A)-L_{mod1}H_{N}H_{CN}). The sequence corresponds to SEQ ID NO. 2 wherein the amino acid residues 1096-1296 are deleted.

SEQ ID NO. 7 is an example of a nucleic acid (DNA) that codes for a recombinant botulinum neurotoxins type B with loop sequence modified according to the invention and C-terminal hexahistidine tag (rBoTN(B)-mod1).

SEQ ID NO. 8 is an example for a recombinant botulinum neurotoxin type B with loop sequence modified according to the invention and C-terminal hexahistidine tag (rBoTN(B)-mod1).

SEQ ID NO. 9 is an example of a nucleic acid (DNA) that codes for a recombinant LH_{N} fragment of the botulinum neurotoxins type B with loop sequence modified according to the invention and C-terminal hexahistidine tag (rBoTN(B)-L_{mod1}H_{N}). The sequence corresponds to SEQ ID NO. 7 wherein the nucleotides 2623-3915 have been deleted.

SEQ ID NO. 10 is an example of a recombinant LH_{N} fragment of the botulinum neurotoxins type B with loop sequence modified according to the invention and C-terminal hexahistidine tag (rBoTN(B)-L_{mod1}H_{N}). The sequence corresponds to SEQ ID NO. 8 wherein the amino acid residues 875-1305 are deleted.

SEQ ID NO. 11 is an example for a nucleic acid (DNA) that codes for a recombinant botulinum neurotoxin type C1 with loop sequence modified according to the invention and C-terminal hexahistidine tag (rBoTN(C1)-mod1).

SEQ ID NO. 12 is an example of a recombinant botulinum neurotoxins type C1 with loop sequence modified according to the invention and C-terminal hexahistidine tag (rBoTN(C1)-mod1).

SEQ ID NO. 13 is an example of a nucleic acid (DNA) that codes for a recombinant LH_{N} fragment of the botulinum neurotoxin type C1 with loop sequence modified according to the invention and C-terminal hexahistidine tag (rBoTN(C1)-L_{mod1}H_{N}). The sequence corresponds to SEQ ID NO. 11 wherein the nucleotides 2599-3858 are deleted.

SEQ ID NO. 14 is an example of a recombinant LH_{N} fragment of the botulinum neurotoxin type C1 with loop sequence modified according to the invention and C-terminal hexahistidine tag (rBoTN(C1)-L_{mod1}H_{N}). The sequence corresponds to SEQ ID NO. 12 wherein the amino acid residues 867-1286 are deleted.

SEQ ID NO. 15 is an example of a nucleic acid (DNA) that codes for a recombinant botulinum neurotoxin type E with loop sequence modified according to the invention and C-terminal hexahistidine tag (rBoTN(E)-mod1).

SEQ ID NO. 16 is an example for a recombinant botulinum neurotoxin type E with loop sequence modified according to the invention and C-terminal hexahistidine tag (rBoTN(E)-mod1).

SEQ ID NO. 17 is an example of a nucleic acid (DNA) that codes for a recombinant 40 kDa fragment of pseudomonas exotoxin comprising the domains II, Ib, and III with loop sequence modified according to the invention and C-terminal hexahistidine tag (PE40-mod1).

SEQ ID NO. 18 is an example of a recombinant 40 kDa fragment of pseudomonas exotoxin comprising the domains II, Ib, and III with loop sequence modified according to the invention and C-terminal hexahistidine tag (PE40-mod1).

SEQ ID NO. 19 is an example of a nucleic acid (DNA) that codes for a recombinant fragment of the diphtheria toxin comprising the A-chain and an N-terminal fragment of the B-chain with loop sequence modified according to the invention and C-terminal hexahistidine tag (DT389-mod1).

SEQ ID NO. 20 is an example of a recombinant fragment of the diphtheria toxin comprising the A-chain and an N-terminal fragment of the B-chain with loop sequence modified according to the invention and C-terminal hexahistidine tag (DT389-mod1).

SEQ ID NO. 21 is an example of a nucleic acid (DNA) that codes for a recombinant ricin toxin with loop sequence modified according to the invention and C-terminal hexahistidine tag (rRicin-mod1).

SEQ ID NO. 22 is an example of a recombinant ricin toxin with loop sequence modified according to the invention and C-terminal hexahistidine tag (rRicin-mod1).

### EXAMPLES

### Example 1

### Cloning and Expression of the LH_{N} Fragment of Botulinum Neurotoxin Type A with Modified Loop

For cloning the DNA sequences of the light chain as well as of the translocation domain, chromosomal DNA was isolated from a culture of Clostridium botulinum type A (strain ATCC 3502). By PCR amplification with primers # 1 and # 2 (FIG. 6) a gene fragment coding for the light chain of BoNT(A) with modified loop sequence and C-terminal His tag was obtained. The PCR amplification product was cloned into the expression plasmid pQE-60 via restriction sites for Nco 1 and Sal 1 so that the plasmid pQE-BoNT(A)-L_{mod1} resulted. By PCR amplification with the primers # 3 and # 4 (FIG. 6) the gene fragment coding for the translocation domain of BoNT(A) was generated. Via the restriction sites for Stu I and Xho I it was cloned between the loop sequence and the sequence for the His tag in pQE-BoNT(A)-L_{mod1} (plasmid pQE-BoNT(A)-L_{mod1}H_{N}; sequence # 2, FIG. 3, No. 2). The E. coli expression strain M15[pREP4] (Qiagen) was transformed with the plasmid pQE-BoNT(A)-L_{mod1}H_{N}. The expression of the modified LH_{N} fragment was realized by a stepped induction with 500 M final concentration IPTG at 25 degrees Celsius over night. The cells were lysed in a 50 mM phosphate buffer at pH 8.0 with 300 mM NaCl by lysozyme treatment and ultrasound treatment. The centrifuged lysate was chromatographed on a Ni-NTA agarose column. An analysis on SDS polyacrylamide gel showed that under reducing conditions two bands at approximately 50 kDA as well as a band at 100 kDA were stained by Coomassie while under non-reducing conditions only the band at 100 kDa was observed (FIG. 7). In this way, it is unequivocally demonstrated that the LH_{N} fragment was released from the bacteria to more than 75 percent as a dichain polypeptide in which the two chains are covalently bonded to one another by a disulfide bridge. The subsequent treatment with thrombin resulted, on the one hand, in cleavage of the single-chain form and, on the other hand, in shortening of the translocation domain in the dichain polypeptide (FIG. 7). A two-hour incubation of the E. coli lysate before purification of the LH_{N} fragment resulted with complete cleavage in the dichain polypeptide.

A correspondingly expressed and purified LH_{N} fragment with the native loop sequence (FIG. 3, No. 1) showed on SDS polyacrylamide gel under non-reducing as well as under reducing conditions a band at 100 kDa. The single-chain polypeptide could be converted only upon cleavage with trypsin into the two-chain disulfide-bridged LH_{N} fragment.

### Example 2

### Cloning and Expression of the LH_{N}H_{CN} Fragment of Botulinum Neurotoxin Type A with Modified Loop and Characterization of the Cleavage Site

The H_{N}H_{CN} fragment (translocation domain with N-terminal half of receptor binding domain of BoNT(A)) was generated by PCR amplification with the primers # 3 and # 5 (FIG. 6) and cloned via restriction sites for Stu I and Xho I into the plasmid pQE-BoNT(A)-L_{mod1} (plasmid pQE-BoNT(A)-L_{mod1}H_{N}H_{CN}; sequence # 3). Expression and purification were carried out in accordance with the scheme described in example 1. An analysis on SDS polyacrylamide gel showed in addition to a weak band that corresponded to the single-chain polypeptide and further undefined bands, a band at 50 kDa as well as one at 75 kDa that corresponded to the light chain and the H_{N}H_{CN} fragment. The N-terminal sequencing of the first four amino acid residues of the H_{N}H_{CN} fragment provided the sequence Ser-Leu-Val-Pro. The cleavage by protease activity in E. coli lysate took place thus after Lys₄₄₀ and thus N-terminally of the pentapeptide Val-Pro-Arg-Gly-Ser inserted into the loop.

### Example 3

### Cloning and Expression of the LH_{N} Fragment of Botulinum Neurotoxin Type B with Modified Loop

For cloning the DNA sequences of the light chain as well as the translocation domain, chromosomal DNA was isolated from a culture of Clostridium botulinum type B (strain Okra). By PCR amplification with the primers # 6 and # 7 (FIG. 6) a gene fragment was generated that codes for the light chain of BoNT(B) with modified loop sequence of BoNT(A). With primers # 8 and # 9 (FIG. 6) a gene fragment coding for the translocation domain of BoNT(B) was generated. Cloning into the expression plasmid pQE-60 was realized first by exchange of the BoNT(A)-L gene fragment in pQE-BoNT(A)-L_{mod1} for the BoNT(B)-L_{mod1} amplification product via the restriction sites for Nco I and Stu I. Subsequently, the BoNT(B)-H_{N} amplification product was cloned therebehind via the restriction sites for Stu I and Xho I so that the plasmid pQE-BoNT(B)-L_{mod1}H_{N} resulted (sequence # 5). The expression in the host strain M15[pREP4] and the purification of the LH_{N} fragment were realized in analogy to example 1. Analysis on SDS polyacrylamide gel showed that under reducing conditions two bands at approximately 50 kDa and 55 kDa were stained by Coomassie while under non-reducing conditions a band at approximately 105 kDa was observed (FIG. 8). These shows unequivocally that the LH_{N} fragment was released from the bacteria substantially as a dichain polypeptide in which the two chains to more than 80 percent were covalently linked with one another by a disulfide bridge.

### Example 4

### Cloning and Expression of the LH_{N} Fragment of the Botulinum Neurotoxin Type C1 with Modified Loop and Characterization of the Cleavage Site

For cloning the DNA sequences of the light chain as well as of the translocation domain, chromosomal DNA was prepared from a culture of Clostridium botulinum type C1 (strain C205). By PCR amplification with the primers # 10 and # 11 (FIG. 6) a gene fragment was generated that codes for the light chain of BoNT(C1) with modified loop sequence of BoNT(A). With primers # 12 and # 13 (FIG. 6) the gene fragment coding for the translocation domain of BoNT(C1) was generated. Cloning into the expression plasmid pQE-60 was realized first by exchange of the BoNT(A)-L gene fragment in pQE-BoNT(A)-L_{mod1} for the pQE-BoNT(C1)-L_{mod1} amplification product via the restriction sites for Nco I and Stu I. Subsequently, the BoNT(C1)-H_{N} amplification product was cloned therebehind via the restriction sites for Stu I and Xho I so that the plasmid pQE-BoNT(C1)-L_{mod1}H_{N} resulted (sequence # 7). The expression in the host strain M15[pREP4] and the purification of the LH_{N} fragment was realized in analogy to example 1. Analysis on SDS polyacrylamide gel showed that under reducing conditions two bands at approximately 50 kDa and 55 kDa were stained by Coomassie while under non-reducing conditions a band at approximately 105 kDa was observed. This shows unequivocally that the LH_{N} fragment was released from the bacteria to more than 90 percent as a dichain polypeptide in which the two chains are covalently linked with one another by a disulfide bridge. The N-terminal sequencing of the first four amino acid residues of the H_{N} fragment resulted in the sequence Ser-Leu-Val-Pro. The cleavage by protease activity in E. coli lysate occurred behind Lys₄₄₇ and thus N-terminal of the pentapeptide Val-Pro-Arg-Gly-Ser inserted into the BoNT(A) loop. By means of directed mutagenesis the arginine residue of the inserted pentapeptide was exchanged for histidine, tyrosine, and glutamine. The mutagenized LH_{N} fragments expressed in the same way were present after two hours of incubation of the E. coli lysate to more than 90 percent in the dichain disulfide-bridged form wherein the efficiency of the cleavage is slightly less than for the LH_{N} fragment that contains the BoNT(A) loop modified with the pentapeptide Val-Pro-Arg-Gly-Ser.

### Example 5

### Cloning and Expression of a Recombinant Botulinum Neurotoxin Type C1 with Modified Loop

By employing chromosomal DNA of the strain Clostridium botulinum C205 the gene fragment coding for the heavy chain was amplified with the primers # 12 and # 14 (FIG. 6). Via the restriction sites for Stu I and Xho I it was cloned into the plasmid BoNT(C1)-L_{mod1}H_{N} between the sequence section coding for the light chain and the sequence for the His tag (plasmid pQE-BoNT(C1)-L_{mod1}H_{N}H_{C}; sequence # 6). The E. coli expression strain M15[pREP4] (Qiagen) was transformed with the corresponding expression plasmid. The expression in the host strain M15[pREP4] and the purification was carried out in analogy to example 1. An analysis on SDS polyacrylamide gel showed that under reducing conditions two bands at approximately 50 kDa and at 105 kDA were stained by Coomassie while under non-reducing conditions only a band at approximately 155 kDa was observed (FIG. 9). In this way, it is unequivocally demonstrated that the recombinant neurotoxin was released from the bacteria to more than 90 percent as a dichain polypeptide in which the two chains were covalently linked to one another by a disulfide bridge. An activity test in the hemidiaphragm assay resulted in a toxicity that is comparably high as that of the native neurotoxin type C1 isolated from Clostridium botulinum. The modification of the loop area between the light chain and the translocation domain therefore had no effect on the toxicity.

### Example 6

### Cloning and Expression of a Recombinant Fragment of the Pseudomonas Exotoxin (Pe40) with Modified Loop

By employing chromosomal DNA of the strain Pseudomonas aeruginosa 103, a gene fragment, coding for the area of the domain II that is boated C-terminally of the loop between the cysteine residues 13 and 36 as well as for the domain III, was amplified by means of PCR with the primers # 17 and # 18 (FIG. 6). The amplification product was cloned into the plasmid pQE-BoNT(A)-L_{mod1} via Nco I and Mlu I in exchange for the gene fragment BoNT(A)-L_{mod1} (plasmid pQE-PEII₃ III). The sequence section for the area of the domain II that is N-terminal of the loop was inserted by hybridization of the oligonucleotide # 15 and # 16 (FIG. 6) and cloning via restriction sites for Nco I and Kpn I into the plasmid pQE-PEII₃ III (plasmid pQE-PEII_{mod} III; sequence # 9). The E. coli expression strain M15[pREP4] (Qiagen) was transformed by the corresponding expression plasmid. The expression in the host strain M15[pREP4] and the purification are carried out in analogy to example 1. An analysis on SDS gel under reducing conditions resulted in a weaker band at 40 kDa as well as a stronger one at 37 kDa. Under non-reducing conditions, however, only one band at 40 kDa was observed. When incubating the cell lysate for at least two hours at room temperature before purification by affinity chromatography, the band at 40 kDa was no longer detectable under reducing conditions. By the exchange of the loop area between the cysteine residues 13 and 36 in domain II of the PE40 fragrant for a modified BoNT(A) loop, cleavage of the polypeptide chain thus occurred wherein the aforementioned cysteine residues formed a disulfide bridge. The N-terminal fragment of approximately 3 kDa was no longer detected after reduction in 12 percent SDS gel.

### Example 7

### Cloning and Expression of a Recombinant Fragment of the Diphtheria Toxin (Dt389) with Modified Loop

By employing chromosomal DNA of the strain Corynebacterium diphtheria NCTC 13129 the gene fragment that codes for the A-chain of the diphtheria toxin was amplified by PCR with the primers # 19 and # 20 (FIG. 6). Via the restriction sites for Nco I and Stu I the amplification product was cloned into the plasmid pQE-BoNT(A)-L_{mod1} (see example 1) (plasmid pQE-DT-A_{mod1}). In the same way, the gene fragment coding for the N-terminal fragment of the B-chain was amplified with the primers # 21 and # 22 (FIG. 6) and cloned via the restriction sites for Stu I and Xho I into pQE-DT-A_{mod1} (plasmid (plasmid pQE-DT389-_{mod1}; sequence # 10). The E. coli expression strain M15[pREP4] (Qiagen) was transformed by the corresponding expression plasmid. The expression in the host strain M15[pREP4] and the purification are carried out in analogy to example 1. An analysis on SDS polyacrylamide gel showed that under reducing conditions two bands at approximately 22 kDa were stained by Coomassie while under non-reducing conditions one band at approximately 43 kDa was observed. This shows unequivocally that the recombinant diphtheria toxin fragment is released from the bacteria to more than 90 percent as a dichain polypeptide in which the two chains are covalently linked with one another by a disulfide bridge.

### Example 8

### Cloning and Expression of Recombinant Ricin with Modified Loop

By employing mRNA of seeds of Ricinus communis the gene fragment coding for the A-chain of ricin was amplified by means of RT-PCR with the primers # 23 and # 24 (FIG. 6). Via the restrictions sites for Nco I and Xho I it was cloned into the plasmid pQE-BoNT(A)-L_{mod1} (see example 1) (plasmid pQE-ricin-A). In the same way the gene fragment coding for the B-chain was amplified with the primers # 25 and # 26 (FIG. 6) and cloned into the pQE-ricin-A via the restriction sites for Kpn I and Xho I (plasmid pQE-ricin-mod1; sequence # 11). The E. coli expression strain M15[pREP4] (Qiagen) was transformed by the corresponding expression plasmid. The expression in the host strain M15[pREP4] and the purification of the soluble portion of the expressed ricin were carried out in analogy to example 1. An analysis on SDS polyacrylamide gel showed that under reducing conditions two bands at approximately 19 kDa and 42 kDa were stained by Coomassie while under non-reducing conditions a band at approximately 62 kDa was observed. This shows unequivocally that the soluble portion of the recombinant ricin is released from the bacteria to more than 90 percent as a dichain polypeptide in which the two chains are covalently linked with one another by a disulfide bridge.

### SCIENTIFIC LITERATURE

Arora et al. (1999), Cancer Res. 59:183-8
Collier (2001), Toxicon 39 (11): 1793-803
Fujinaga (1997), Microbiology 143: 3841-47
Ogata et al. (1990), J. Biol. Chem. 265(33): 20678-85
Reiter (2001), Adv. Cancer Res. 81: 93-124
Schiavo and Montecucco (1997), The Clostridia: Molecular Biology and Pathogenesis, Academic Press, San Diego: 295-322
Williams et al. (1990) J. Biol Chem, 265(33): 20673-77

### PATENT LITERATURE

Borgford, U.S. Pat. No. 6,593,132
Brown and Jones, WO 89/04839
Fitzgerald et al., U.S. Pat. No. 6,426,075
Pastan et al., U.S. Pat. No. 5,980,895

What is claimed is:

1.-42. (canceled)

43. A method for producing polypeptides or proteins in a dichain form comprised of a first chain and a second chain, wherein the first and second chains are disulfide-bridged, by recombinant expression in E. coli host cells, wherein
(i) the polypeptide or protein exerts a biologic activity as a dichain disulfide-bridged polypeptide or protein,
(ii) a C-terminal amino acid residue of the first chain is an Arg residue or Lys residue,
(iii) the second chain of the protein/polypeptide has N-terminally 1 to 20 amino acid residues and a pentapeptide PRS sequence VPXGS, wherein X is any naturally occurring amino acid, wherein V is Val, Leu, Ile, Ala, Phe, Pro or Gly, wherein P is Pro, Leu, Ile, Ala, Phe, Val or Gly, wherein G is Gly, Leu, Ile, Ala, Pro, Phe or Val, and wherein S is Ser, Tyr, Trp, or Thr; and
(iv) the method comprises the steps of:
(a) modifying the polypeptide or protein, at the nucleic acid level, to a modified construct so that the polypeptide or protein in said modified construct in a loop area has a PRS sequence VPXGS wherein X, V, P, G, and S are as defined above;
(b) inserting said modified construct into E. coli host cells;
(c) cultivating and subsequently lysing the host cells; and
(d) isolating the dichain disulfide-bridged peptide or protein.

44. The method according to claim 43, wherein the first chain of the polypeptide or protein is the light chain of the polypeptide or protein and the second chain is the heavy chain of the polypeptide or protein.

45. The method according to claim 44, wherein the polypeptide or protein is a botulinum neurotoxin.

46. The method according to claim 43, wherein the polypeptide or protein is the botulinum neurotoxin of the serotype A (BoNT(A)) or the LH_{N} fragment of BoNT(A).

47. The method according to claim 46, wherein the PRS sequence VPXGS is inserted between the amino acids Leu₄₄₂ and Lys₄₄₈ of BoNT(A) with deletion of the amino acids 443-447.

48. The method according to claim 47, wherein the PRS sequence VPXGS is selected from the group consisting of VPRGS, VPYGS, VPHGS, and VPQGS.

49. The method according to claims 43, wherein the PRS sequence VPXGS is inserted into the octapeptide Lys₄₃₈-Ile₄₄₅ of BoNT(B), into the 15mer His₄₃₈-Asp₄₅₂ of BoNT(C1) or into the 13mer Lys₄₁₃-Ile₄₂₅ of BoNT(E) with deletion of at least one amino acid.

50. The method according to claim 49, wherein the PRS sequence VPXGS is inserted in the form of the 17mer GIITSKTKSLVPRGSKA or the 18mer RGIITSKTKSLVPRGSKA.

51. The method according to claim 43, wherein the protein is a hybrid protein.

52. The method according to claim 51, wherein the hybrid protein has the following components A, B, and C:
an effector domain having enzymatic activity and the enzymatic activity enables the effector domain to inhibit secretion in target cells or kill the target cells, or a toxin domain (component A);
a loop sequence that comprises the PRS sequence VPXGS (component B); as well as
a cell binding domain that imparts a cell specificity to the fusion protein or hybrid protein (component C).

53. The method according to claim 52, wherein the hybrid protein additionally comprises a component D as a translocation domain.

54. The method according to claim 52, wherein the toxin domain (component A) is selected from the group of the domain of the diphtheria toxin, the domain of the pseudomonas exotoxin, and the domain of ricin.

55. The method according to claim 54, wherein the toxin domain (component A) is the fragment PE40 (domain III, domain II and domain Ib) or the fragment PE38 (domain III and domain II) of the pseudomonas exotoxin or the A-chain of ricin.

56. The method according to claim 52, wherein the cell binding domain (component C) is a monoclonal antibody, an affilin, an ankyrin repeat protein, an anticalin, a growth factor, or a cytokine.

57. The method according to claim 56, wherein the growth factor is selected from the group consisting of TGF-alpha, FGF, VEGF, and IGF-1; and wherein the cytokine is selected from the group consisting of IL2, IL4, and IL6.

58. The method according to claim 51 wherein the hybrid protein has the following components A, B, and C:
a protein or an oligo peptide that imparts to the fusion protein at least one of a better solubility, a higher expression rate, and capability of affinity purification (component A);
a loop sequence comprising the sequence VPXGS (component B), and
any type of polypeptide (component C).

59. The method according to claim 58, wherein the component A is glutathione-S-transferase (GST), a maltose binding protein (MBP), a His tag, a StrepTag, or a FLAG tag.

60. The method according to claim 43, wherein the E. coli host cells are E. coli K12 cells.

61. The method according to claim 43, wherein the E. coli host cells are E. coli K12 cells of the strains M15[pREP4], XL1-BLUE, or UT5600.

62. A polypeptide/protein, wherein the polypeptide/protein is present as a dichain disulfide-bridged polypeptide/protein comprised of a first chain and a second chain and wherein the polypeptide/protein is biologically active, wherein the C-terminal end of the first chain of the polypeptide/protein is an Arg residue or Lys residue and the second chain of the polypeptide/protein comprises N-terminally 1 to 20 amino acid residues and a pentapeptide PRS sequence VPXGS, wherein X is any naturally occurring amino acid, wherein V is Val, Leu, Ile, Ala, Phe, Pro or Gly, wherein P is Pro, Leu, Ile, Ala, Phe, Val or Gly, wherein G is Gly, Leu, Ile, Ala, Pro, Phe or Val, and wherein S is Ser, Tyr, Trp, or Thr.

63. The polypeptide/protein according to claim 62, wherein the first chain of the polypeptide/protein is the light chain of the polypeptide/protein and the second chain is the heavy chain of the polypeptide/protein.

64. The polypeptide/protein according to claim 62, wherein the C-terminal end of the first chain is a Lys residue.

65. The polypeptide/protein according to claim 62, wherein the second chain has N-terminally the pentapeptide sequence VPXGS, the hexapeptide sequence XVPXGS, or the heptapeptide sequence XXVPXGS.

66. The polypeptide/protein according to claim 62, wherein the polypeptide/protein is a botulinum neurotoxin, a derivative of a botulinum neurotoxin or a fragment of botulinum neurotoxin or has the biologic activity of a botulinum neurotoxin.

67. The polypeptide/protein according to claim 66, wherein the fragment is the LH_{N} fragment.

68. The polypeptide/protein according to claim 62, wherein the polypeptide/protein is the botulinum neurotoxin of the serotype A (BoNT(A)) or has the biologic activity of BoNT(A).

69. The polypeptide/protein according to claim 62, wherein the polypeptide/protein is the LH_{N} fragment of BoNT(A) or has the biologic activity of BoNT(A).

70. The polypeptide/protein according to claim 62, wherein the second chain has N-terminally the heptapeptide sequence SLVPXGS.

71. The polypeptide/protein according to claim 70 wherein X is R, Y, H, or Q.

72. The polypeptide/protein according to claim 62, wherein the protein is a hybrid protein.

73. The polypeptide/protein according to claim 72, wherein the hybrid protein has the following components A, B, and C:
an effector domain having enzymatic activity and the enzymatic activity enables the effector domain to inhibit secretion in target cells or kill the target cells, or a toxin domain (component A);
a loop sequence that comprises the PRS sequence VPXGS (component B); and
a cell binding domain that imparts a cell specificity to the protein (component C).

74. The polypeptide/protein according to claim 73, wherein the hybrid protein additionally has a translocation domain as a component D.

75. The polypeptide/protein according to claim 73, wherein the toxin domain (component A) is selected from the group consisting of the domain of the diphtheria toxin, the domain of the pseudomonas exotoxin, and the domain of ricin.

76. The polypeptide/protein according to claim 75, wherein the toxin domain (component A) is the fragment PE40 (domain III, domain II and domain Ib) or the fragment PE38 (domain III and domain II) of the pseudomonas exotoxin or the A-chain of ricin.

77. The polypeptide/protein according to claim 73, wherein the cell binding domain (component C) is a monoclonal antibody, an affilin, an ankyrin repeat protein, an anticalin, a growth factor, or a cytokine.

78. The polypeptide/protein according to claim 77, wherein the growth factor is selected from the group consisting of TGF-alpha, FGF, VEGF, and IGF-1; and wherein the cytokine is selected from the group consisting of IL2, IL4, and IL6.

79. The polypeptide/protein according to claim 72, wherein the hybrid protein has the following components A, B, and C:
a protein or an oligo peptide that imparts to the fusion protein at least one of a better solubility, a higher expression rate, and capability of affinity purification (component A);
a loop sequence comprising the PRS sequence VPXGS (component B); and
any type of polypeptide (component C).

80. The polypeptide/protein according to claim 79, wherein the component A is glutathione-S-transferase (GST), the maltose binding protein (MBP), a His tag, a StrepTag or a FLAG tag.

81. A nucleic acid coding for the polypeptide/protein according to claim 62.

82. The nucleic acid according to claim 81, which is DNA.

83. A vector comprising the nucleic acid according to claim 81.

84. A host cell comprising the vector according to claim 83.

85. The host cell according to claim 84, wherein the host cell is prokaryotic, especially an E coli cell and preferred an E. coli K12 cell.

86. The host cell according to claim 84, wherein the host cell is M15[pREP4] and wherein the vector is a plasmid of the pQE series.

87. A pharmaceutical preparation comprising the polypeptide/protein according to claim 62.
